SEQUENCE LISTING (6 sequences)

SEQ ID NO: 1 - DNA, 24 bp, artificial sequence
  synthetic aptamer
  modified_base (1)..(23): all purines (A , G and I) are deoxy, except at positions 1, 10 and 14, wherein adenosine is 2′-O-methyl, and at positions 2, 7 and 17, wherein guanosine is 2′-O-methyl; all pyrimidines (C and U) are 2′-O-methyl
  misc_feature (8)..(9): deoxy guanosine at position 8 is linked to deoxy guanosine at position 9 via a phosphorothioate internucleotide linkage
  misc_feature (15)..(16): 2′-O-methyl uridine at position 15 is linked to deoxy inosine at position 16 via a phosphorothioate internucleotide linkage
  modified_base (16)..(16): I
  misc_feature (16)..(17): deoxy inosine at position 16 is linked to 2′-O-methyl guanosine at position 17 via a phosphorothioate internucleotide linkage
  modified_base (18)..(18): I
  misc_feature (18)..(19): deoxy inosine at position 18 is linked to deoxy guanosine at position 19 via a phosphorothioate internucleotide linkage
  modified_base (20)..(20): I
  misc_feature (20)..(21): deoxy inosine at position 20 is linked to deoxy guanosine at position 21 via a phosphorothioate internucleotide linkage
  misc_feature (24)..(24): thymidine at position 24 is an inverted orientation deoxy thymidine (3′-3′ linked)
agccuggggacccaungngngcut

SEQ ID NO: 2 - DNA, 24 bp, artificial sequence
  synthetic aptamer
  modified_base (1)..(23): all purines (A , G and I) are deoxy, except at positions 1, 10 and 14, wherein adenosine is 2′-O-methyl, and at positions 2, 7 and 17, wherein guanosine is 2′-O-methyl; all pyrimidines (C and U) are 2′-O-methyl
  misc_feature (8)..(9): deoxy guanosine at position 8 is linked to deoxy guanosine at position 9 via a phosphorothioate internucleotide linkage
  misc_feature (15)..(16): 2′-O-methyl uridine at position 15 is linked to deoxy inosine at position 16 via a phosphorothioate internucleotide linkage
  modified_base (16)..(16): I
  misc_feature (16)..(17): deoxy inosine at position 16 is linked to 2′-O-methyl guanosine at position 17 via a phosphorothioate internucleotide linkage
  misc_feature (17)..(18): 2′-O-methyl guanosine at position 17 is linked to deoxy inosine at position 18 via a phosphorothioate internucleotide linkage
  modified_base (18)..(18): I
  misc_feature (18)..(19): deoxy inosine at position 18 is linked to deoxy guanosine at position 19 via a phosphorothioate internucleotide linkage
  modified_base (20)..(20): I
  misc_feature (20)..(21): deoxy inosine at position 20 is linked to deoxy guanosine at position 21 via a phosphorothioate internucleotide linkage
  misc_feature (24)..(24): thymidine at position 24 is an inverted orientation deoxy thymidine (3′-3′ linked)
agccuggggacccaungngngcut

SEQ ID NO: 3 - DNA, 24 bp, artificial sequence
  synthetic aptamer
  modified_base (1)..(23): all purines (A , G and I) are deoxy, except at positions 1, 10 and 14, wherein adenosine is 2′-O-methyl, and at positions 2, 7 and 17, wherein guanosine is 2′-O-methyl; all pyrimidines (C and U) are 2′-O-methyl
  misc_feature (8)..(9): deoxy guanosine at position 8 is linked to deoxy guanosine at position 9 via a phosphorothioate internucleotide linkage
  misc_feature (15)..(16): 2′-O-methyl uridine at position 15 is linked to deoxy inosine at position 16 via a phosphorothioate internucleotide linkage
  modified_base (16)..(16): I
  misc_feature (16)..(17): deoxy inosine at position 16 is linked to 2′-O-methyl guanosine at position 17 via a phosphorothioate internucleotide linkage
  modified_base (18)..(18): I
  misc_feature (18)..(19): deoxy inosine at position 18 is linked to deoxy guanosine at position 19 via a phosphorothioate internucleotide linkage
  misc_feature (19)..(20): deoxy guanosine at position 19 is linked to deoxy inosine at position 20 via a phosphorothioate internucleotide linkage
  modified_base (20)..(20): I
  misc_feature (20)..(21): deoxy inosine at position 20 is linked to deoxy guanosine at position 21 via a phosphorothioate internucleotide linkage
  misc_feature (24)..(24): thymidine at position 24 is an inverted orientation deoxy thymidine (3′-3′ linked)
agccuggggacccaungngngcut

SEQ ID NO: 4 - DNA, 24 bp, artificial sequence
  synthetic aptamer
  modified_base (1)..(23): all purines (A , G and I) are deoxy, except at positions 1, 10 and 14, wherein adenosine is 2′-O-methyl, and at positions 2, 7 and 17, wherein guanosine is 2′-O-methyl; all pyrimidines (C and U) are 2′-O-methyl
  misc_feature (8)..(9): deoxy guanosine at position 8 is linked to deoxy guanosine at position 9 via a phosphorothioate internucleotide linkage
  misc_feature (15)..(16): 2′-O-methyl uridine at position 15 is linked to deoxy inosine at position 16 via a phosphorothioate internucleotide linkage
  modified_base (16)..(16): I
  misc_feature (16)..(17): deoxy inosine at position 16 is linked to 2′-O-methyl guanosine at position 17 via a phosphorothioate internucleotide linkage
  misc_feature (17)..(18): 2′-O-methyl guanosine at position 17 is linked to deoxy inosine at position 18 via a phosphorothioate internucleotide linkage
  modified_base (18)..(18): I
  misc_feature (18)..(19): deoxy inosine at position 18 is linked to deoxy guanosine at position 19 via a phosphorothioate internucleotide linkage
  misc_feature (19)..(20): deoxy guanosine at position 19 is linked to deoxy inosine at position 20 via a phosphorothioate internucleotide linkage
  modified_base (20)..(20): I
  misc_feature (20)..(21): deoxy inosine at position 20 is linked to deoxy guanosine at position 21 via a phosphorothioate internucleotide linkage
  misc_feature (24)..(24): thymidine at position 24 is an inverted orientation deoxy thymidine (3′-3′ linked)
agccuggggacccaungngngcut

SEQ ID NO: 5 - DNA, 19 bp, artificial sequence
  synthetic aptamer
  modified_base (1)..(19): all purines (A , G and I) are deoxy, except at positions 1, 10 and 14, wherein adenosine is 2′-O-methyl, and at positions 2, 7 and 17, wherein guanosine is 2′-O-methyl; all pyrimidines (C and U) are 2′-O-methyl
  misc_feature (8)..(9): deoxy guanosine at position 8 is linked to deoxy guanosine at position 9 via a phosphorothioate internucleotide linkage
  misc_feature (15)..(16): 2′-O-methyl uridine at position 15 is linked to deoxy inosine at position 16 via a phosphorothioate internucleotide  linkage
  modified_base (16)..(16): I
  misc_feature (16)..(17): deoxy inosine at position 16 is linked to 2′-O-methyl guanosine at position 17 via a phosphorothioate internucleotide linkage
  modified_base (18)..(18): I
  misc_feature (18)..(19): deoxy inosine at position 18 is linked to deoxy guanosine at position 19 via a phosphorothioate internucleotide linkage
agccuggggacccaungng

SEQ ID NO: 6 - DNA, 17 bp, artificial sequence
  synthetic aptamer
  modified_base (1)..(17): All purines (A , G and I) are deoxy, except at positions 1, 10 and 14, wherein adenosine is 2′-O-methyl, and at positions 2, 7 and 17, wherein guanosine is 2′-O-methyl; all pyrimidines (C and U) are 2′-O-methyl
  misc_feature (8)..(9): deoxy guanosine at position 8 is linked to deoxy guanosine at position 9 via a phosphorothioate internucleotide linkage
  misc_feature (15)..(16): 2′-O-methyl uridine at position 15 is linked to deoxy inosine at position 16 via a phosphorothioate internucleotide linkage
  modified_base (16)..(16): I
  misc_feature (16)..(17): deoxy inosine at position 16 is linked to 2′-O-methyl guanosine at position 17 via a phosphorothioate internucleotide linkage
agccuggggacccaung

(19) United States
(12) Patent Application Publication
(10) Pub. No.: US 2009/0075342 A1
(43) Pub. Date: Mar. 19, 2009

(54) Metabolic profile directed aptamer medicinal chemistry

(76) Inventors: Sharon Cload, Cambridge, MA (US); Christine P. Donahue, Brighton, MA (US); Renta Hutabarat, Winchester, MA (US); Kristin Thompson, Arlington, MA (US); Charles Wilson, Concord, MA (US)

Correspondence Address: MINTZ, LEVIN, COHN, FERRIS, GLOVSKYAND POPEO, P.C., ONE FINANCIAL CENTER, BOSTON, MA, 02111, US

(21) Appl. No.: 12/150,855

(22) Filed: Apr. 30, 2008

Related U.S. Application Data
(63) Continuation-in-part of application No. 11/115,780, filed on Apr. 26, 2005.
(60) Provisional application No. 60/927,061, filed on Apr. 30, 2007.
(60) Provisional application No. 60/998,769, filed on Oct. 12, 2007.
(60) Provisional application No. 61/065,520, filed on Feb. 12, 2008.

(51) Int. Cl. C07H 19/00; C12P 19/30

(52) U.S. Cl. 435/89; 536/22.1; 536/28.4; 536/27.21

(57) ABSTRACT

The present invention provides materials and methods for enhancing aptamers. More specifically, the materials and methods of the present invention are directed toward the modification of aptamers by the identification of one or more cleavage sites, and introduction of a chemical substitution at a position proximal to the cleavage site(s). Such aptamers are useful for the treatment of disease, in diagnostic and detection applications, and/or research, e.g., target validation.

### CROSS-REFERENCE TO RELATED APPLICATIONS

This non-provisional patent application claims priority under 35 U.S.C. § 120 to and is a continuation-in-part of U.S. patent application Ser. No. 11/115,780, filed Apr. 26, 2005, currently pending, and under 35 U.S.C. § 119(e) to U.S. Provisional Patent Application Ser. No. 60/927,061, filed Apr. 30, 2007; U.S. Provisional Patent Application Ser. No. 60/998,769, filed Oct. 12, 2007; and U.S. Provisional Patent Application Ser. No. 61/065,520, filed Feb. 12, 2008; each of which is herein incorporated by reference in its entirety.

### FIELD OF INVENTION

The invention relates generally to the field of nucleic acids, and more particularly to aptamers useful as therapeutics, diagnostics and in research, such as for target validation. The invention further relates to materials and methods for enhancing aptamers for use in therapeutics, diagnostics and research.

### BACKGROUND OF THE INVENTION

Aptamers are nucleic acid molecules having specific binding affinity to molecules through interactions other than classic Watson-Crick base pairing.

Aptamers, like peptides generated by phage display or monoclonal antibodies (“mAbs”), are capable of specifically binding to selected targets and modulating the target's activity or binding interactions, e.g., through binding an aptamer may block or activate its target's ability to function. Discovered by an in vitro selection process from pools of random sequence oligonucleotides, aptamers have been generated for hundreds of proteins, including growth factors, transcription factors, enzymes, immunoglobulins and receptors. A typical aptamer is 10-15 kDa in size (20-45 nucleotides), binds its target with nanomolar to sub-nanomolar affinity, and discriminates against closely related targets (e.g., aptamers will typically not bind other proteins from the same gene family). A series of structural studies have shown that aptamers are capable of using the same types of binding interactions (e.g., hydrogen bonding, electrostatic complementarities, hydrophobic contacts and steric exclusion) that drive affinity and specificity in antibody-antigen complexes.

Aptamers have a number of desirable characteristics for use as therapeutics and diagnostics including high specificity and affinity, biological efficacy and excellent pharmacokinetic properties. In addition, they offer specific competitive advantages over antibodies and other protein biologics.

Furthermore, the aptamer discovery process readily permits modification, such as aptamer sequence optimization and the minimization of aptamer length. See, e.g., Conrad et al. 1996; Eaton et al. 1997; Cload et al., The Aptamer Handbook, 363-416 (2006); and Wilson et al., Curr Opin Chem Biol, 10(6), 607-614 (2006). Additionally, 2′-modifications, such as 2′-fluoro and 2′-O-Me, may be utilized for stabilization against nucleases without compromising the aptamer binding interaction with the target. See, e.g., Lin et al., Nucleic Acids Res., 22, 5229-5234 (1994); Jellinek et al., Biochemistry, 34, 11363-1137 (1995); Lin et al., Nucleic Acids Res., 22, 5229-5234 (1994); Kubik et al., J. Immunol., 159(1), 259-267 (1997); Pagratis et al., Nat. Biotechnol., 1, 68-73 (1997); and Wilson et al., Curr Opin Chem Biol, 10(6), 607-614 (2006).

Chemical substitutions have been incorporated into libraries of transcripts from which aptamers are discovered with the view towards selecting aptamers with various characteristics, such as increased target affinity. See, e.g., Latham et al., Nucleic Acids Res., 22, 2817-2822 (1994); Vaish et al., Biochemistry, 42, 8842-8851 (2003); Saitoh et al., Nucleic Acids Res. Suppl., 2, 215-216 (2002); Masud et al., Bioorg. Med. Chem., 12, 1111-1120 (2004); King et al., Biochemistry, 41, 9696-9706 (2002); and Yang, X. and Gorenstein, D. G., Curr. Drug Targets, 5, 705-715 (2004). However, introduction of substitutions into libraries of transcripts via transcription is a “global” approach in which all nucleotides of a given kind are simultaneously substituted. This “global” approach does not allow for the discovery of single substitutions that increase a desired aptamer characteristic, e.g., aptamer-target affinity or metabolic stability, but may not be tolerated at other positions within the aptamer.

It would be beneficial to alter a desired aptamer characteristic, for example, metabolic stability, including resistance to endonucleases and exonucleases, while minimizing the number of chemical substitutions required to do so, as well as the efforts needed to identify such substitutions. The present invention provides materials and methods to meet these and other needs.

### SUMMARY OF THE INVENTION

The present invention provides materials and methods related to the identification and substitution of aptamers for use in the treatment of disease, diagnostic and detection applications, and research applications, such as target validation.

One embodiment of the invention is a method for identifying and modifying an aptamer comprising the steps of: a) incubating a parent aptamer with a test fluid to result in a mixture; b) analyzing the mixture to identify metabolites of the parent aptamer, thereby detecting at least one aptamer cleavage site in the parent aptamer; and c) introducing a chemical substitution at a position proximal to the at least one aptamer cleavage site to result in a modified aptamer. Another embodiment of the invention is a method for enhancing the stability of an aptamer comprising the steps above. A further embodiment of the invention is a method for enhancing the stability of an aptamer to exonucleases and/or endonucleases comprising the steps above.

In some embodiments of the invention, the test fluid is a biological matrix, particularly a biological matrix selected from the group consisting of one or more of: serum; plasma; cerebral spinal fluid; tissue extracts, including cytosolic fraction, S9 fraction and microsomal fraction; aqueous humour; vitreous humour and tissue homogenates. In some embodiments, the biological matrix is derived from a species selected from the group consisting of one or more of: mouse, rat, monkey, pig, human, dog, guinea pig and rabbit. In some embodiments, the test fluid comprises at least one purified enzyme, particularly at least one purified enzyme selected from the group consisting of: snake venom phosphodiesterase and DNAse 1.

In some embodiments of the invention, the analyzing step comprises analyzing the resulting aptamer using liquid chromatography and mass spectrometry, particularly electron spray ionization liquid chromatography mass spectrometry, polyacrylamide gel electrophoresis or capillary electrophoresis to determine a position of at least one aptamer cleavage site. In some embodiments, the analyzing step comprises analyzing the resulting aptamer using a bioanalytical method selected from the group consisting of one or more of: denaturing polyacrylamide gel electrophoresis (PAGE); capillary electrophoresis; HPLC and LC/MS, particularly LC/MS/MS or LC/MS/MS/MS, and more particularly ESI-LC/MS, ESI-LC/MS/MS and ESI-LC/MS/MS/MS.

In some embodiments, the proximal position comprises a position selected from the group consisting of: a position immediately 5′ to the aptamer cleavage site, a 5′ position at or within three nucleotides of the aptamer cleavage site, a position immediately 3′ to the aptamer cleavage site, a 3′ position at or within three nucleotides of the aptamer cleavage site, and at the cleaved internucleotide linkage.

In some embodiments, the chemical substitution is selected from the group consisting of: a chemical substitution at a sugar position; a chemical substitution at a base position and a chemical substitution at a phosphate position. More particularly, a substitution is selected from the group consisting of: a purine substitution for a pyrimidine; a 2′-deoxy dihydrouridine substitution for a uridine; a 2′-deoxy-5-methyl cytidine for a cytidine; a 2-amino purine substitution for a purine; a phosphorothioate substituted for a phosphodiester; a phosphorodithioate substituted for a phosphodiester; a deoxynucleotide substituted for a 2′-OH nucleotide; a 2′-OMe nucleotide, a 2′-fluoro nucleotide or a 2′-O-methoxyethyl nucleotide substituted for a 2′-OH or deoxynucleotide; and the addition of a PEG polymer.

In additional embodiments, the introducing step further comprises introducing more than one chemical substitution at one or more cleavage sites or at a single cleavage site or both. In another embodiment of the invention, wherein more than one aptamer cleavage site is detected, the introducing step further comprises introducing at least one chemical substitution at the associated proximal position of the aptamer cleavage site determined to occur first in time during the incubating step or at any other cleavage site(s) that provides the desired properties upon introduction of a chemical substitution.

In other embodiments, the method further comprises the step of testing the stability of the modified aptamer in the test fluid. In some embodiments of the invention, aptamer stability is assessed by determining the percent of modified aptamer that remains intact in the test fluid as compared to the percent of the parent aptamer that remains intact in the test fluid. In some embodiments, the percent of intact aptamer is assessed by a bioanalytical method selected from the group consisting of one or more of: denaturing polyacrylamide gel electrophoresis (PAGE); capillary electrophoresis; HPLC and LC/MS, particularly LC/MS/MS or LC/MS/MS/MS, and more particularly ESI-LC/MS, ESI-LC/MS/MS and ESI-LC/MS/MS/MS. In other embodiments, the modified aptamer is more stable in the test fluid than the parent aptamer, preferably at least 2 fold, more preferably at least 5 fold and most preferably at least 10 fold more stable.

In additional embodiments of the invention, the method further comprises determining a dissociation constant of the modified aptamer for its target. In some embodiments, chemical substitutions are introduced singly at each position or in various combinations in the aptamer, and the dissociation constant for each resulting aptamer is determined. Chemical substitutions are introduced at a position proximal to the aptamer cleavage site such that a single chemical modification results in a dissociation constant for the modified aptamer that is the same or less than that of the parent aptamer. In another embodiment of the invention, the method comprises selecting a modified aptamer having a dissociation constant for its target that is the same or less than that for the parent aptamer.

In other embodiments, the modified aptamer binds to a target having a biological activity, and the method further comprises testing the biological activity of the target in the presence and absence of modified aptamer. In another embodiment, the method further comprises selecting a modified aptamer that binds to a target having a biological activity that is the same or better than that of the parent aptamer. The biological activity may be measured in any relevant assay, such as an ELISA assay or a cell-based assay.

In some embodiments, the incubating, analyzing, introducing and testing steps are repeated iteratively until the desired stability is achieved.

### BRIEF DESCRIPTION OF THE DRAWINGS

FIG. 1 is a schematic representation of the in vitro aptamer selection (SELEX™) process from pools of random sequence oligonucleotides.

FIG. 2 is a schematic representation of the process for optimizing aptamer stability in a biological matrix that includes recombinant or purified enzymes from various species incubated at various times and temperatures.

FIG. 3 is an illustration of a 40 kDa branched PEG.

FIG. 4 is an illustration of a 40 kDa branched PEG that is attached to the 5′ end of an aptamer.

FIG. 5 is an illustration depicting various PEGylation strategies representing standard mono-PEGylation, multiple PEGylation and oligomerization via PEGylation.

FIG. 6A is a table illustrating the masses of the major metabolites of ARC1666 observed after incubating the aptamers in pooled cynomolgus macaque serum for various times. FIG. 6B is an illustration of the putative secondary structure of ARC1666 with the major nuclease cleavage products highlighted as Metabolite 1 and Metabolite 2. Underlined nucleotides contain a 2′-O-methyl ribose, and all other nucleotides contain a 2′-deoxyribose. Asterisks between nucleotides represent phosphorothioate linkages.

FIG. 7 is a bar graph of the percent of ARC1666 and its derivatives remaining after incubation in cynomolgus monkey serum and arrows represent the major nuclease cleavage sites.

FIG. 8 shows detailed chromatograms of ARC1666, ARC2982, ARC2983 and ARC3901 (top to bottom) following 48 hour incubation in cynomolgus monkey serum.

FIG. 9 (A and B) shows graphs of the percent of intact (parent aptamer) ARC3901 remaining in monkey, rat and human serum (vertical axis) as detected by ESI-LC/MS and plotted as a function of incubation time (horizontal axis).

FIG. 10 is a series of three tables illustrating the masses of the major metabolites of ARC3901 observed after incubating the aptamers for various times in either pooled human, cynomolgus macaque or rat serum.

FIG. 11 is a line plot representing the relative intensity and migration distance of bands in the gel from the 24 hour time point after incubation in rat serum for ARC1666, ARC2982, ARC2983 and ARC3901.

FIG. 12 is a bar plot representing the relative percentage of full length aptamer remaining after 24 hours in rat serum for ARC1666, ARC2982, ARC2983 and ARC3901.

FIG. 13 is a line plot representing the relative mobilities of cleavage products of ARCXXXA as assessed via PAGE.

FIG. 14 shows graphs of the percent full length aptamer remaining in human and rat serum, based upon SAX-HPLC analyses, and plotted as a function of time for ACRXXXA, ARCXXXB and ARCXXXC.

FIG. 15 shows detailed chromatograms of ARCXXXA, ARCXXXB and ARCXXXC (top to bottom) following 48 hour incubation in rat and human serum.

FIG. 16 shows graphs of the percent full length aptamer remaining in rat and human serum, based upon SAX-HPLC analyses, and plotted as a function of time for ARCXXXA.

FIG. 17 is a detailed chromatogram of ARCXXXA following 120 hour incubation in human and rat serum.

FIG. 18 shows graphs of the percent full length aptamer remaining in rat and human serum, based upon SAX-HPLC analyses, and plotted as a function of time for ARCXXXB, ARCXXXC and ARCXXXD.

FIG. 19 shows detailed chromatograms of ARCXXXB, ARCXXXC and ARCXXXD (top to bottom) following 121 hour incubation in human and rat serum.

FIG. 20 shows graphs of the percent full length aptamer remaining in rat and human serum, based upon SAX-HPLC analyses, and plotted as a function of time for ACRXXXA, ARCXXXB, ARCXXXC, ARCXXXD, ARCXXXE and ARCXXXF.

FIG. 21 shows detailed chromatograms of ARCXXXA, ARCXXXB, ARCXXXC, ARCXXXD, ARCXXXE and ARCXXXF (top to bottom) following 48 hour incubation in rat serum.

FIG. 22 shows detailed chromatograms of ARCXXXA, ARCXXXB, ARCXXXC, ARCXXXD, ARCXXXE and ARCXXXF (top to bottom) following 48 hour incubation in human serum.

FIG. 23 shows graphs of the percent full length aptamer remaining in rat and human serum, based upon SAX-HPLC analyses, and plotted as a function of time for ACRXXXA, ARCXXXO, ARCXXXS, ARCXXXT, ARCXXXK, ARCXXXL, ARCXXXI and ARCXXXJ.

FIG. 24 shows detailed chromatograms of ARCXXXA, ARCXXXO, ARCXXXS, ARCXXXL and ARCXXXI (top to bottom) following 48 hour incubation in rat, monkey, human and dog serum.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present specification will control.

### Aptamer Development

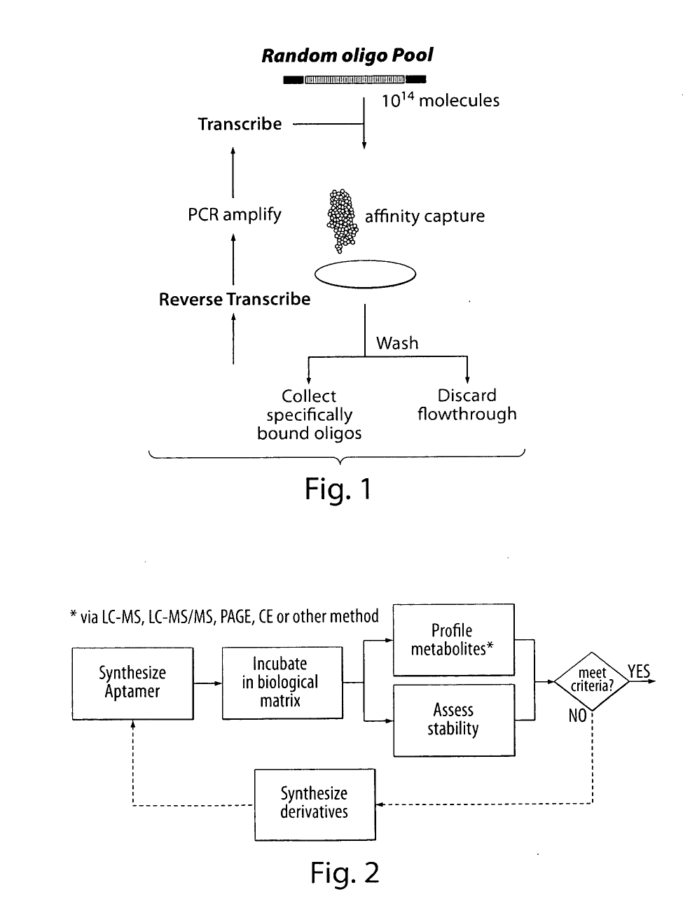

An aptamer, also referred to herein as a nucleic acid ligand, comprises an isolated nucleic acid molecule having specific binding affinity to a molecule through interactions other than classic Watson-Crick base pairing. A suitable method for identifying an aptamer is with the process entitled “Systematic Evolution of Ligands by Exponential Enrichment” (“SELEX™”), which is generally depicted in FIG. 1 and described in the literature. Once aptamers for use, e.g., therapeutic and/or diagnostic leads and/or for target validation, have been identified, the aptamers may be modified to achieve desired criteria.

### Aptamer Medicinal Chemistry

Aptamer medicinal chemistry is used to improve aptamer characteristics, such as to achieve a particular, e.g., therapeutic, criteria. Aptamer medicinal chemistry is performed following selection of the aptamer of interest, and typically following the optional minimization and mutagenesis steps described in the literature.

In one embodiment of the invention, aptamer medicinal chemistry uses a strategy in which sets of variant aptamers are chemically synthesized. These sets of variants typically differ from the parent aptamer by the substitution of a single nucleotide or other residue for a starting nucleotide or other residue. The substituted nucleotide or other residue differs from the one it is replacing by at least one chemical modification. In the context of a nucleotide, the chemical modification may occur at the nucleotide base, sugar or phosphate position. In the methods of the present invention where the chemical modification is at a base position in a nucleotide, the chemical modification does not result in the interconversion of one nucleotide for another within the group of A, U, G or C, or within the group A, T, G or C.

Within the set of variant aptamers, the variant aptamers differ from each other by the location of the substituted nucleotide or other residue (“substituent”). These variants are then compared to each other and to the parent. Where the variants are compared to the parent with regards to binding affinity for the aptamer target, the structure activity relationship (“SAR”) may be determined. Improvements in characteristics, particularly binding affinity, may be profound enough that the inclusion of a single substituent may be all that is necessary to achieve a particular therapeutic criterion.

Alternatively, the information gleaned from the set of single variants may be used to design further sets of variants in which more than one substituted nucleotide or other residue is introduced simultaneously. In one design strategy, all of the single substituent variants are ranked based upon the improvement conferred by the single substituent on therapeutic criteria, the top 4 are chosen and all possible double (6), triple (4) and quadruple (1) combinations of these 4 single substituent variants are synthesized and assayed. In a second design strategy, the best single substituent variant is considered to be the new parent and all possible double substituent variants that include this highest-ranked single substituent variant are synthesized and assayed. In another design strategy, single substituent variants in which the substitution did not significantly adversely affect binding affinity are combined with other single substitution variants, synthesized and assayed. Furthermore, single substituent variants that do not significantly adversely effect binding affinity and/or those that increased binding affinity may be combined with a second type of substitution, synthesized and assayed. For example, an inosine substitution may be combined with a substitution of a 2′-deoxy nucleotide for a 2′-OMe nucleotide to arrive at a variant having higher affinity relative to the unsubstituted starting aptamer or either singly substituted parent.

Other strategies may be used, and these strategies may be applied repeatedly such that the number of substituents is gradually increased while continuing to identify further-improved variants. For example, some substitution strategies use block substitutions. Particularly, the secondary structure of the aptamer may be predicted, and based upon predicted secondary structure, blocks of nucleotides from the parent aptamer may be replaced with modified blocks of nucleotides. For example, where the predicted secondary structure comprises a stem loop structure, the nucleotide blocks comprised in the predicted stem may be replaced with modified nucleotides (e.g. 2′-OMe nucleotides) as well as those completing the loop. Blocks that increase affinity are retained. Blocks that do not increase affinity may be further characterized using the single substitution strategy within that block region.

In one embodiment, stabilizing substitutions, e.g., 2′-OMe substitutions for nuclease resistance, may be introduced into an aptamer that actually reduce the binding affinity of the substituted aptamer relative to the unsubstituted starting aptamer. A second substitution, e.g., a phosphorothioate substitution, may be introduced into the substituted aptamer and assayed for binding affinity equivalent to or better than that of the unsubstituted starting aptamer.

Aptamer medicinal chemistry may be used particularly as a method to explore the local, rather than the global, introduction of substituents. Because aptamers are discovered within libraries that are generated by transcription, any substituents that are introduced during the SELEX™ process must be introduced globally. For example, if it is desired to introduce phosphorothioate linkages between nucleotides, then they can only be introduced at every A (or every G, C, T, U, etc.) (globally substituted). Aptamers that require phosphorothioates at some A's (or some G, C, T, U, etc.) (locally substituted) to achieve a desired therapeutic criteria, but cannot tolerate it at other A's, cannot be readily discovered by this process.

The types of substituents that can be utilized by the aptamer medicinal chemistry process are not limited to nucleotides alone, rather they are substituents that may be generated as solid-phase synthesis reagents and are capable of introduction into an oligomer synthesis scheme. Aptamer medicinal chemistry schemes may include substituents that introduce steric bulk, hydrophobicity, hydrophilicity, lipophilicity, lipophobicity, positive charge, negative charge, neutral charge, zwitterions, polarizability, nuclease-resistance, conformational, rigidity, conformational flexibility, protein-binding characteristics, mass, etc. Aptamer medicinal chemistry schemes may include base-modifications, sugar-modifications or phosphodiester linkage-modifications.

When considering the kinds of substituents that are likely to be beneficial within the context of a therapeutic aptamer, it may be preferred to introduce substitutions that fall into one or more of the following categories:

(1) substituents that are naturally occurring, e.g., 2′-deoxy, 2′-ribo, 2′-OMe purines or pyrimidines, or 2′-deoxy-5-methyl cytidine;
(2) substituents already part of an approved therapeutic, e.g., phosphorothioate-linked oligonucleotides; or
(3) substituents that hydrolyze or degrade to one of the above two categories, e.g., methylphosphonate-linked oligonucleotides.

### Metabolic Profile Directed Aptamer Medicinal Chemistry

Aptamer medicinal chemistry may also be used to enhance the stability of aptamers, such as resistance to exonucleases and endonucleases, which can metabolize aptamers in test fluids, biological matrices, serum and tissues.

In general, a method of the invention involves incubating a parent aptamer with a test fluid to result in a mixture. Then, the mixture is analyzed in order to determine the rate of disappearance of the parent aptamer, the specific aptamer metabolic profile and the specific aptamer metabolite sequences. Knowledge of the sequences of the specific metabolites formed allows one to infer the sites of nuclease cleavage. After systematically conducting metabolic profiling and identifying specific aptamer cleavage sites, the method involves introducing chemical substitutions or modifications at or near the cleavage sites that are designed to block nuclease cleavage. Another embodiment of the invention is a method for enhancing the stability of an aptamer comprising the steps above. A further embodiment of the invention is a method for enhancing the stability of an aptamer to exonucleases and/or endonucleases comprising the steps above.

The first step of the methods involves incubating a parent aptamer in one or more test fluids to result in a mixture.

In general, the methods involve incubating at least 10 nM of parent aptamer in the one or more test fluids. Preferably, the methods involve incubating 1-100 μM of parent aptamer in the one or more test fluids. Most preferably, the methods involve incubating 5-30 μM of parent aptamer in the one or more test fluids.

In general, the methods involve incubating the parent aptamer between 4-50° C. Preferably, the parent aptamer is incubated between 20-40° C. Most preferably, the parent aptamer is incubated at 37° C.

In general, the methods involve incubating the parent aptamer for 0-150 hours. Preferably, the parent aptamer is incubated for 2-120 hours.

Only one test fluid may be used in the methods. Alternatively, more than one test fluid may be used in the methods, either in combination or sequentially. The test fluid may be any fluid. Examples of test fluids include, but are not limited to, a low pH fluid (pH of 1-4 or 1-7), a high pH fluid (pH of 7-10 or 7-14), a room temperature or body temperature fluid, or a biological matrix. Preferably, the test fluid is a biological matrix. Preferably, the biological matrix is selected from the group consisting of one or more of: serum; plasma; cerebral spinal fluid; tissue extracts, including cytosolic fraction, S9 fraction and microsomal fraction; aqueous humour; vitreous humour and tissue homogenates. Preferably, the biological matrix is derived from a species selected from the group consisting of one or more of: mouse, rat, monkey, pig, human, dog, guinea pig and rabbit. Preferably, the test fluid comprises at least one purified enzyme. More preferably, the enzyme is selected from the group consisting of snake venom phosphodiesterase and DNAse 1.

Suitable incubation conditions for a specific purpose may be readily determined by a person having ordinary skill in the art using known methods.

The second step of the methods involves analyzing the mixture to identify metabolites of the parent aptamer, thereby detecting at least one aptamer cleavage site in the parent aptamer. The analyzing step may be performed by any one or more bioanalytical methods known in the art. Examples of such bioanalytical methods include, but are not limited to, denaturing polyacrylamide gel electrophoresis, capillary electrophoresis, HPLC, LC/MS, LC/MS/MS, LC/MS/MS/MS, ESI-LC/MS, ESI-LC/MS/MS and ESI-LC/MS/MS/MS. Preferably, the analyzing step comprises analyzing the mixture using liquid chromatography and mass spectrometry. Alternatively, the analyzing step comprises analyzing the mixture using electron spray ionization liquid chromatography mass spectrometry, polyacrylamide gel electrophoresis or capillary electrophoresis to determine a position of at least one aptamer cleavage site, preferably all cleavage sites.

For analysis, the samples may be extracted by any liquid organic extraction method or solid phase extraction method. Alternatively, the samples may be treated with Proteinase K and incubated at 55° C. overnight with shaking.

The digested samples are then centrifuged and the supernatant is taken and analyzed. Preferably, the samples are centrifuged between 10,000-25,000 rpm. Most preferably, the samples are centrifuged at 14,000 rpm. Preferably, the samples are centrifuged between 4-15° C. Most preferably, the samples are centrifuged at 4° C. Preferably, the samples are centrifuged for 1-20 minutes. Most preferably, the samples are centrifuged for 12 minutes.

Suitable analysis conditions for a specific purpose may be readily determined by a person having ordinary skill in the art using known methods.

Using the results of the metabolite profiling assay, along with SAR data obtained independently as described above, the medicinal chemist may design aptamer derivatives that contain substitutions designed to block sites of metabolism (nuclease cleavage), preferably major sites of metabolism, or any other cleavage site that provides an aptamer with the desired properties. A major site of cleavage (metabolism) is time-dependent and exhibits more significant cleavage or proportionately greater cleavage relative to a minor site of cleavage, which can be determined readily by someone of ordinary skill in the art. Basically, a major site of cleavage is differentiated from a minor site of cleavage based upon the time of cleavage and the amount of cleavage, both of which are readily determined by someone of ordinary skill in the art. The aptamer analogues may again be evaluated for metabolic stability in order to confirm that nuclease resistance has been achieved (FIG. 2).

In the methods of the invention, chemical substitutions or modifications are introduced into aptamers at a position proximal to the cleavage site(s). The proximal position is either at or near the cleavage site(s) in either two-dimensional (linear) or three-dimensional space. In two-dimensional space, the proximal position is: a position immediately 5′ to the aptamer cleavage site, a 5′ position at or within three nucleotides of the aptamer cleavage site, a position immediately 3′ to the aptamer cleavage site, a 3′ position at or within three nucleotides of the aptamer cleavage site, or a position at the cleaved internucleotide linkage. In three-dimensional space, the proximal position may be located anywhere along the linear molecule, but will be located at or near the cleavage site when the aptamer is folded in a three-dimensional configuration. Preferably, in linear space, the proximal position is a position immediately 5′ to the aptamer cleavage site, a position immediately 3′ to the aptamer cleavage site or a position at the cleaved internucleotide linkage. More preferably, the proximal position is at the cleaved internucleotide linkage and the chemical substitution is at a phosphate position; the proximal position is 5′ or 3′ to the aptamer cleavage site and the chemical substitution is at a sugar or base position; or a combination thereof. In addition, a large moiety, such as a PEG moiety, could be located far from a nuclease cleavage site yet still block nuclease cleavage.

Typical chemical modifications (substitutions) that block nuclease cleavage include a chemical substitution at a sugar, phosphate or base position, such as substituting the phosphodiester linkage at the cleavage site with a phosphorothioate linkage, addition of a 2′-O-methyl group on the base 5′ or 3′ to the cleaved phosphodiester linkage or a combination thereof. In addition, a 3′ cap or 5′ cap, such as an inverted dT (inverted deoxy thymidine) or a 3′ amine on the phosphate, may be used to block nuclease cleavage. The creation of a series of analogues is aimed at, for example, improving the metabolic stability, improving the distribution phase, improving the effectiveness or increasing the bioavailability of the drug. This optimization stage aims to develop new aptamers that are more effective than the early aptamers. The optimization cycle can be done more than once until the desired optimized aptamer is identified. This metabolic stability optimization cycle or process may be repeated to create a stabilized aptamer that will suit its therapeutic purpose.

Preferably, a chemical substitution is selected from the group consisting of: a purine substitution for a pyrimidine; a 2′-deoxy dihydrouridine substitution for a uridine; a 2′-deoxy-5-methyl cytidine for a cytidine; a 2-amino purine substitution for a purine; a phosphorothioate substituted for a phosphodiester; a phosphorodithioate substituted for a phosphodiester; a deoxynucleotide substituted for a 2′-OH nucleotide; a 2′-OMe nucleotide, a 2′-fluoro nucleotide or a 2′-O-methoxyethyl nucleotide substituted for a 2′-OH or deoxynucleotide; the addition of a PEG or PAG polymer; the addition of a large steric molecule; the addition of a 3′ cap; and any other modification known to block nuclease degradation.

In additional embodiments, the introducing step further comprises introducing more than one chemical substitution at one or more cleavage sites or into a single cleavage site or both. In another embodiment of the invention, wherein more than one aptamer cleavage site is detected, the introducing step further comprises introducing at least one chemical substitution at the associated proximal position of the aptamer cleavage site determined to occur first in time during the incubating step. However, a chemical substitution can be made at any of the cleavage sites, and not just the first in time to occur.

Preferably, the modified aptamer binds a target with the same or better affinity than the parent aptamer. Binding affinity may be measured by any method known in the art, such as a Biacore assay.

In additional embodiments of the invention, the method further comprises the step of testing the stability of the modified aptamer in the test fluid. In further embodiments, the method comprises repeating the incubating, analyzing, introducing and testing steps iteratively. Preferably, the modified aptamer is more stable in the test fluid than the parent aptamer. Preferably, stability is assessed by the percent of modified aptamer that remains intact in the test fluid as compared to the percent of the parent aptamer that remains intact in the test fluid. Preferably, the percent of intact aptamer is assessed by one or more bioanalytical methods, such as denaturing polyacrylamide gel electrophoresis, capillary electrophoresis, HPLC, LC/MS, LC/MS/MS, LC/MS/MS/MS, ESI-LC/MS, ESI-LC/MS/MS or ESI-LC/MS/MS/MS.

In other embodiments, the method further comprises the step of determining a dissociation constant of the modified aptamer for a target. A dissociation constant may be determined by any method known in the art. In further embodiments, the method comprises selecting a modified aptamer having a dissociation constant for its target that is the same or less than a dissociation constant of the parent aptamer.

In additional embodiments of the invention, the modified aptamer binds a target having a biological activity and the method further comprises the step of measuring the biological activity of the target in the presence and absence of aptamer. Biological activity may be measured by any method known in the art. Preferably, the biological activity is measured by an ELISA assay or a cell-based assay.

For example, a specific embodiment of the method involves incubation of the test aptamer in a biological matrix (e.g., from rat, mouse, monkey, dog or human) at 37° C. at a concentration of, for example, 5 to 30 μM for a defined period (typically 2 hour to 120 hours), followed by analyzing (profiling) by HPLC and ESI/LC/MS to identify the parent, specific metabolites and specific cleavage sites within the aptamer (FIG. 2). For analysis, the samples are treated with Proteinase K and incubated at 55° C. overnight with shaking. Digested samples are then centrifuged and the supernatant is taken after spinning the samples at 14,000 rpm at 4° C. for 12 minutes and analyzed by HPLC and ESI/LC/MS.

The aptamers of the present invention, including chemically substituted aptamers, can be synthesized using any oligonucleotide synthesis techniques known in the art including solid phase oligonucleotide synthesis techniques (see, e.g., Froehler et al., Nucl. Acid Res., 14:5399-5467 (1986) and Froehler et al., Tet. Lett., 27:5575-5578 (1986)) and solution phase methods, such as triester synthesis methods (see, e.g., Sood et al., Nucl. Acid Res., 4:2557 (1977) and Hirose et al., Tet. Lett., 28:2449 (1978)).

### PEG-Derivatized Nucleic Acids

As described above, derivatization of nucleic acids with high molecular weight non-immunogenic polymers may be used to provide increased resistance to degradation by nucleases.

The aptamer compositions of the invention may be derivatized with polyalkylene glycol (“PAG”) moieties. Examples of PAG-derivatized nucleic acids are found in U.S. Patent Application Publication Number US 20070009476 A1, which is herein incorporated by reference in its entirety. Typical polymers used in the invention include polyethylene glycol (“PEG”), also known as polyethylene oxide (“PEO”), and polypropylene glycol (including poly isopropylene glycol). Additionally, random or block copolymers of different alkylene oxides (e.g., ethylene oxide and propylene oxide) can be used in many applications. In its most common form, a polyalkylene glycol, such as PEG, is a linear polymer terminated at each end with hydroxyl groups: HO—CH₂CH₂O—(CH₂CH₂O)ₙ—CH₂CH₂—OH. This polymer, alpha-, omega-dihydroxylpolyethylene glycol, can also be represented as HO-PEG-OH, where it is understood that the—PEG-symbol represents the following structural unit: —CH₂CH₂O—(CH₂CH₂O)ₙ—CH₂CH₂—, where n typically ranges from about 4 to about 10,000.

As shown, the PEG molecule is di-functional and is sometimes referred to as “PEG diol”. The terminal portions of the PEG molecule are relatively non-reactive hydroxyl moieties, the —OH groups, that can be activated or converted to functional moieties for attachment of the PEG to other compounds at reactive sites on the compounds. Such activated PEG diols are referred to herein as bi-activated PEGs. For example, the terminal moieties of PEG diol have been functionalized as active carbonate ester for selective reaction with amino moieties by substitution of the relatively non-reactive hydroxyl moieties, —OH, with succinimidyl active ester moieties from N-hydroxy succinimide.

In many applications, it is desirable to cap the PEG molecule on one end with an essentially non-reactive moiety so that the PEG molecule is mono-functional (or mono-activated). In the case of protein therapeutics that generally display multiple reaction sites for activated PEGs, bi-functional activated PEGs lead to extensive cross-linking, yielding poorly functional aggregates. To generate mono-activated PEGs, one hydroxyl moiety on the terminus of the PEG diol molecule typically is substituted with a non-reactive methoxy end moiety, —OCH₃. The other, un-capped terminus of the PEG molecule typically is converted to a reactive end moiety that can be activated for attachment at a reactive site on a surface or a molecule, such as a protein.

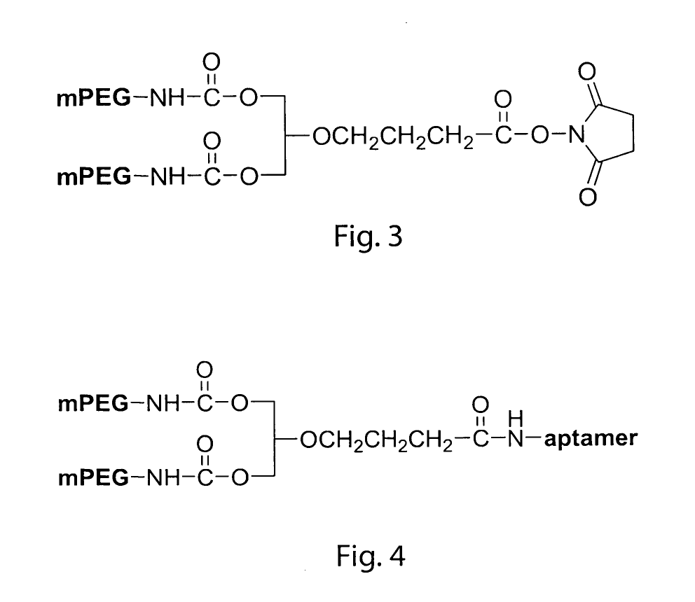

Polyalkylated compounds of the invention are typically between 5 and 80 kDa in size, however any size can be used, the choice dependent on the aptamer and application. Other PAG compounds of the invention are between 10 and 80 kDa in size. Still other PAG compounds of the invention are between 10 and 60 kDa in size. For example, a PAG polymer may be at least 0.01, 0.1, 0.5, 1, 10, 20, 30, 40, 50, 60 or 80 kDa in size. Such polymers can be linear or branched. In some embodiments, the polymers are PEG. In some embodiment, the polymers are branched PEG (see, e.g., FIGS. 3 and 4).

In contrast to biologically-expressed protein therapeutics, nucleic acid therapeutics are typically chemically synthesized from activated monomer nucleotides. PEG-nucleic acid conjugates may be prepared by incorporating the PEG using the same iterative monomer synthesis. For example, PEGs activated by conversion to a phosphoramidite form can be incorporated into solid-phase oligonucleotide synthesis. Alternatively, oligonucleotide synthesis can be completed with site-specific incorporation of a reactive PEG attachment site. Most commonly, this has been accomplished by addition of a free primary amine at the 5′-terminus (incorporated using a modifier phosphoramidite in the last coupling step of solid phase synthesis). Using this approach, a reactive PEG (e.g., one which is activated so that it will react and form a bond with an amine) is combined with the purified oligonucleotide and the coupling reaction is carried out in solution.

### PAG-Derivatization of a Reactive Nucleic Acid

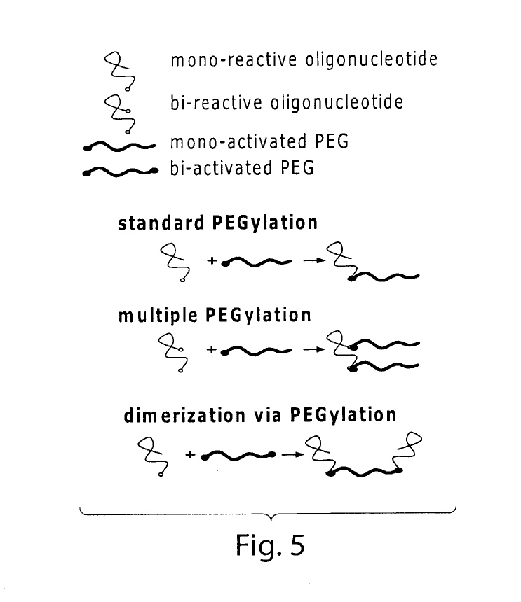

High molecular weight PAG-nucleic acid-PAG conjugates can be prepared by reaction of a mono-functional activated PEG with a nucleic acid containing more than one reactive site. In one embodiment, the nucleic acid is bi-reactive, or bi-activated, and contains two reactive sites: a 5′-amino group and a 3′-amino group introduced into the oligonucleotide through conventional phosphoramidite synthesis, for example: 3′-5′-di-PEGylation, as illustrated in FIG. 5. In alternative embodiments, reactive sites can be introduced at internal positions, using for example, the 5-position of pyrimidines, the 8-position of purines or the 2′-position of ribose as sites for attachment of primary amines. In such embodiments, the nucleic acid can have several activated or reactive sites and is said to be multiply activated. Following synthesis and purification, the modified oligonucleotide is combined with the mono-activated PEG under conditions that promote selective reaction with the oligonucleotide reactive sites while minimizing spontaneous hydrolysis. In the preferred embodiment, monomethoxy-PEG is activated with succinimidyl propionate and the coupled reaction is carried out at pH 8.3. To drive synthesis of the bi-substituted PEG, stoichiometric excess PEG is provided relative to the oligonucleotide. Following reaction, the PEG-nucleic acid conjugate is purified by gel electrophoresis or liquid chromatography to separate fully, partially and un-conjugated species.

The linking domains can also have one or more polyalkylene glycol moieties attached thereto. Such PAGs can be of varying lengths and may be used in appropriate combinations to achieve the desired molecular weight of the composition.

The effect of a particular linker can be influenced by both its chemical composition and length. A linker that is too long, too short, or forms unfavorable steric and/or ionic interactions with the target will preclude the formation of a complex between the aptamer and the target. A linker that is longer than necessary to span the distance between nucleic acids may reduce binding stability by diminishing the effective concentration of the ligand. Thus, it is often necessary to optimize linker compositions and lengths in order to maximize the affinity of an aptamer to a target.

### EXAMPLES

### Example 1

### Metabolic Profile of ARC1666

The metabolic profile of ARC1666 after incubation in pooled cynomolgus monkey serum at a concentration of 50 μM at 37° C. for 0, 2, 8 and 48 hours was determined. At each incubation time point, aliquots were withdrawn, flash-frozen in liquid nitrogen and stored at −80° C. until extraction with Proteinase K. Metabolites were identified after resolving the reaction components by HPLC and subjecting the major peak contents to LC/MS or ESI-LC/MS analysis.

Cynomolgus monkey serum was acquired from Bioreclamation Inc. (Hicksville, N.Y.). A stock solution of ARC1666 was first prepared at 500 μM in Dulbecco's PBS (DPBS: 8.1 mM Na₂HPO₄, 138 mM NaCl, 1.5 mM KH₂PO₄, 2.7 mM KCl, 0.5 mM MgCl₂, and 0.9 mM CaCl₂). Then 100 μL of 500 μM ARC1666 solution was added to 900 μL of cynomolgus monkey serum to make a final stock solution of 50 μM ARC1666 in 90% cynomolgus monkey serum. 180 μL of stock solution was immediately taken out as a 0 hour time point and added to a fresh tube containing 20 μl of 0.5M EDTA in order to block nuclease activity. The rest of the stock solution was immediately put into a 37° C. incubator. Four different time-points (1, 2, 4 and 8 hours) post 37° C. incubation were similarly taken by pipetting out 180 μL stock solution into fresh tubes containing 20 μL of 0.5M EDTA. Tubes containing samples from different time-points were first aliquotted before being frozen at −20° C. for subsequent studies.

Each sample was incubated with 100 μL of Proteinase K Buffer, and 100 μL of Proteinase K (3 mg/mL in Diluent Buffer) was added to each tube containing 200 μL of 50 μM ARC1666 that had been incubated in cynomolgus monkey serum for a specified period of time at 37° C. Each proteinase digestion reaction was incubated overnight at 55° C. with shaking. After incubation, proteinase digestion reaction tubes were centrifuged for 12 minutes at 14,000 rpm and 4° C. Approximately 350 μL of supernatant was collected.

About 50 μL of the Proteinase K extraction solution from each time point was desalted using a CentriSpin 20 Cartridge and filtered through a 0.22 μM spin filter. 30 μL of each filtered sample and an aptamer solution were injected into an Agilent 1100 LC/MS system. The LC separation was based on ion-pair (Hexafluoroisopropanol/Triethylamine) mechanism on a C18 reversed phase column. The chromatogram was monitored by both the UV detector at 260 nm and the single-quadruple mass detector via electro spray interface. The molecular weight of the species under the metabolite peaks were deconvoluted from the total ion current signal of the mass detector. The metabolites were then identified according to their molecular weight.

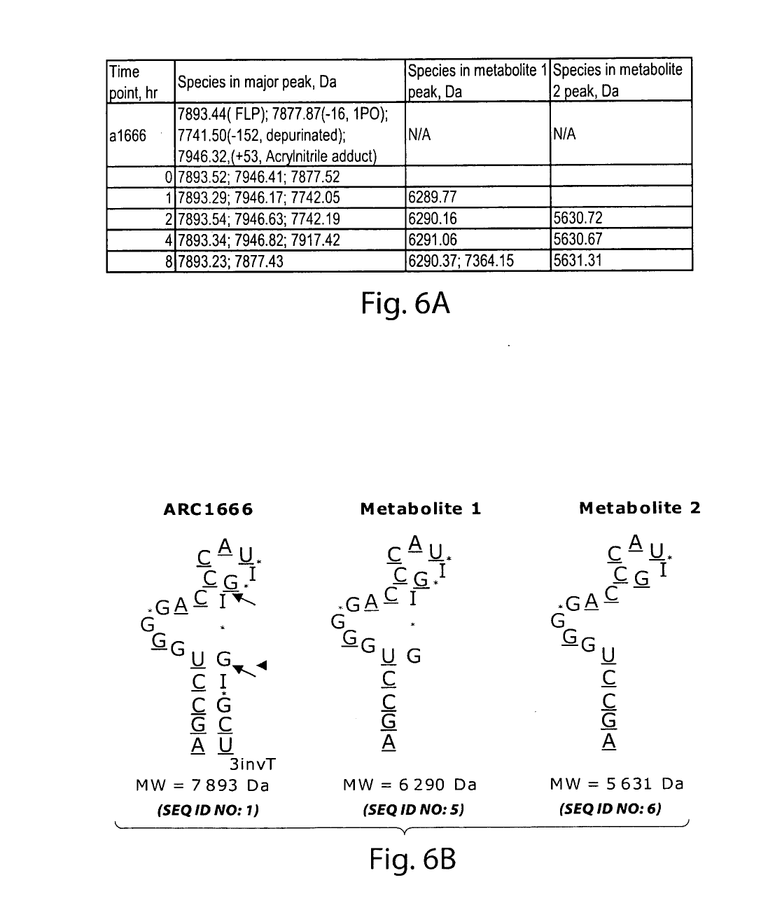

Based upon the metabolic profiling data generated as described above in relation to ARC1666 (FIG. 6A), the primary cleavages appear between residues 17 and 18, and between residues 20 and 21 (FIG. 6B).

### Example 2

### Design of ARC1666 Variants in View of Metabolic Profile Analysis

Based upon the metabolic profile of ARC1666 described in Example 1 above, additional stabilized variants of ARC1666 were identified and tested for potency. Phosphorothioate linkages were introduced between nucleotides. A majority of test variants were tested for h-IgE binding affinity in the dot-blot binding assays previously described as an indicator of relative potency. Chemically synthesized aptamers were purified using denaturing polyacrylamide gel electrophoresis, 5′ end labeled with γ-³²P ATP and were tested for direct binding to full length human h-IgE (Athens Research, Athens, Ga.). A protein titration was used in the dot-blot binding assay in Dulbecco's PBS (with Mg⁺⁺ and Ca⁺⁺) with 0.1 mg/mL BSA at room temperature for 30 minutes. K_{D} values were calculated by fitting the equation y=Max*((([aptamer]+[protein]+K_{D})−SQRT(([aptamer]+[protein]+K_{D})̂2−4([aptamer]*[protein])))/(2*[aptamer]))+y−Int.

Sequences of the ARC1666 derivatives synthesized, purified and assayed for binding to h-IgE, as well as the results of the protein binding characterization are tabulated below in Table 1. As can be seen in Table 1, the binding affinities of the derivatives, are similar to that of ARC1666, indicating that the introduction of additional phosphorothioate linkages did not have an adverse effect on binding affinity.

In Table 1 below, “m” denotes a 2′-O-methyl substituted nucleotide, “d” denotes a deoxyribonucleotide, “s” denotes a phosphorothioate internucleotide linkage and “l” denotes an inosine substitution.

TABLE 1

Sequences and Approximate Binding Affinities of
ARC1666 Derivatives

| | | | |
|---|---|---|---|
| SEQ ID | | | K_{D} |
| NO | ARC# | Sequence | (nM) |

| | | | |
|---|---|---|---|
| 1 | ARC1666 | mAmGmCmCmUdGmGdG-s-dGmAmCmCm | 0.035 |
| | | CmAmU-s-dI-s-mGdI-s-dGdI-s- | |
| | | dGmCmU-3T | |
| 2 | ARC2982 | mAmGmCmCmUdGmGdG-s-dGmAmCmCm | .03 |
| | | CmAmU-s-dI-s-mG-s-dI-s-dGdI- | |
| | | s-dGmCmU-3T | |
| 3 | ARC2983 | mAmGmCmCmUdGmGdG-s-dGmAmCmCm | .02 |
| | | CmAmU-s-dI-s-mGdI-s-dG-s-dI- | |
| | | s-dGmCmU-3T | |
| 4 | ARC3901 | mAmGmCmCmUdGmGdG-s-dGmAmCmCm | 0.13 |
| | | CmAmU-s-dI-s-mG-s-dI-s-dG-s- | |
| | | dI-s-dGmCmU-3T | |

### Example 3

### Metabolic Stability of ARC1666 Derivatives

The metabolic stability of aptamer ARC3901 was evaluated in pooled rat, cynomolgus monkey and human serum at a concentration of approximately 10 μM and incubated at 37° C. for 0, 24, 48, 96 and 120 hours. In addition, the metabolic stabilities of ARC1666, ARC2982 and ARC2983 were evaluated in cynomolgus macaque serum at a concentration, of approximately 10 μM and incubated at 37° C. for 0, 2 and 8 hours. At each incubation time point, aliquots were withdrawn, flash-frozen in liquid nitrogen and stored at −80° C. until extraction with Proteinase K. All samples were analyzed by HPLC and/or ESI-LC/MS for percent full-length aptamer, as well as for metabolite profiling by ESI-LC/MS.

100 μM stocks of HPLC-purified ARC1666, ARC2982, ARC2983 and ARC3901 were prepared in water. Each construct was heated to 99° C. (92° C. measured) for 10 minutes in an Eppendorf Thermoshaker, chilled in an ice bath for 10 min, removed and left at room temperature until use.

The serum incubation assay samples for each species and concentration were prepared as shown below in Table 2 with a total volume of 1 mL. Each sample was mixed briefly and 200 μL aliquots were immediately removed into 1.5 mL tubes and flash-frozen in liquid nitrogen prior to storage at −80° C. for “t=0” samples. From the remaining 800 μL of serum solution, 200 μL of solution was added to fresh tubes labeled by aptamer number (ARC1666, ARC2982, ARC2983 or ARC3901), time point (2, 8 or 48 hours) and serum species, and all were placed in a 37° C. water bath with mild shaking until collection. Upon collection, all time points are immediately flash-frozen in liquid nitrogen and stored at −80° C. until time of analysis.

TABLE 2
Assay Setup

| Reagent | Volume |
|---|---|
| ARCXXXX¹ | 100 |
| Serum² | 900 |

¹10X, 100 μM stocks of ARC1666, ARC2982, ARC2983 and ARC3901
²100% 0.2 μM filtered monkey, human and rat serum (Bioreclamation, Inc.)

Prior to HPLC and ESI-LC/MS analysis, each sample was incubated with 100 μL of Proteinase K Buffer, and 100 μL of Proteinase K (3 mg/mL in Diluent Buffer) was added to each tube containing 200 μL of 10 μM ARC1666, ARC2982, ARC2983 or ARC3901 that had been incubated in serum for a specified period of time at 37° C. Each proteinase digestion reaction was incubated overnight at 55° C. with shaking. After incubation, proteinase digestion reaction tubes were centrifuged for 12 minutes at 14,000 rpm and 4° C. Approximately 350 μL of supernatant was collected from each tube and analyzed via HPLC and/or ESI-LC/MS. Briefly, the SAX-HPLC method may be described as follows:

### Column/Medium: DNAPacPA-100

### Platform/Instrument: Agilent 1100

### Analysis Temperature (C): 80°

### Analyzed Sample Volume (μL): 25

### Solvent A: 25 mM Na Phosphate, pH 7.0, 25% ACN

### Solvent B: 25 mM Na Phosphate, pH 7.0, 25% ACN, 400 mM NaClO₄

### Gradient min (% B): 10

### Gradient max (% B): 65

Gradient Duration (min): 22 minutes

Samples were also analyzed by ESI-LC/MS/MS for metabolite profiling in order to allow determinations of sites of aptamer cleavage. Serum samples for ESI-LC/MS/MS analysis were treated with Proteinase K as described above. Full scan RP-LC/UV/MS data was then collected on ˜100 pmoles of extracted aptamer using an ion trap mass spectrometer with an electrospray ionization source operated in the negative ion mode. Cross-inspection of samples, serum blanks and compound standards were utilized to allow major metabolite peaks to be located using the LC/UV/MS data. Automated data processing software (ProMass Deconvolution™ available from Novatia LLC, Monmouth Junction, N.J.) converted the multiply-charged electrospray mass spectra from selected chromatographic peaks to zero-charge mass spectra. Masses determined from automated data processing were compared with all possible calculated aptamer cleavages retaining either the 5′ or 3′ ends of the molecule. Experimental masses that matched expected cleavage masses were automatically reported as putative metabolites. The disappearance of integrated parent mass intensity can also be used to follow degradation of aptamers over time.

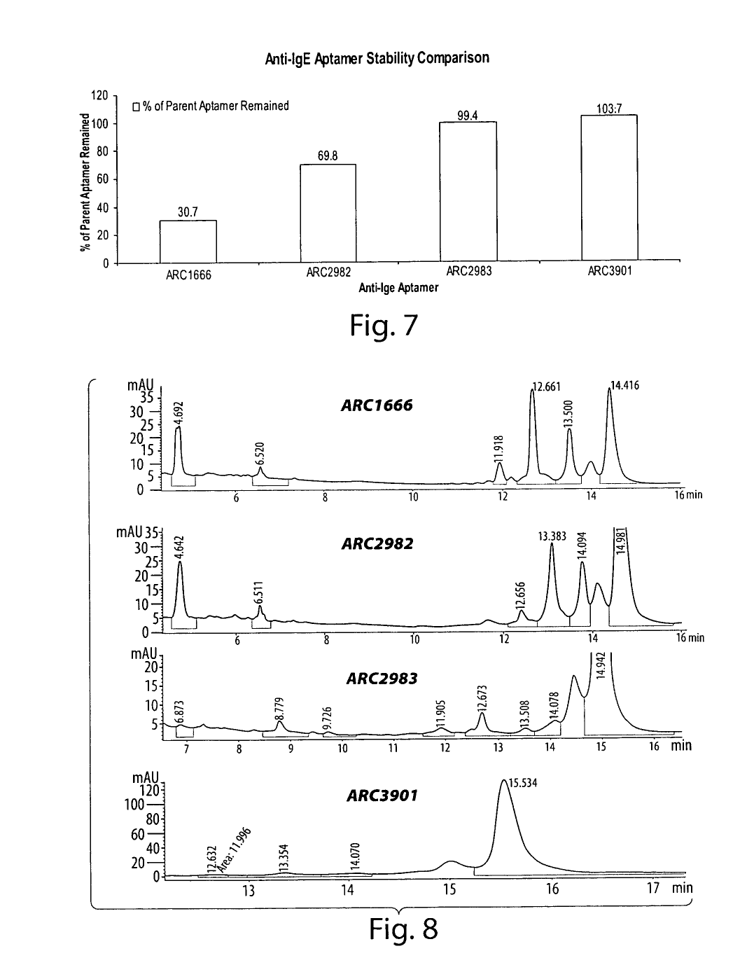

FIG. 7 depicts the percent full length aptamer remaining at 8 hours for ARC1666 and its three derivatives in cynomolgus monkey serum, based upon SAX-HPLC analyses. Detailed SAX-HPLC chromatograms of the aptamers following 8 hour incubation in cynomolgus monkey serum are presented in FIG. 8. Percent of full-length ARC1666, ARC2982, ARC2983 and ARC3901 remaining in cynomolgus monkey serum are reported below in Table 3. Following 8 hour incubations in pooled cynomolgus monkey serum, approximately 31, 70, 99 and 104% of full-length ARC1666, ARC2982, ARC2983 and ARC3901 remained, respectively. The three ARC1666 derivatives have significantly greater metabolic stability than their precursor, ARC1666.

TABLE 3
Percent Full-Length Aptamer Remaining in Cynomolgus Monkey Serum

| Serum Incubation | | | | |
|---|---|---|---|---|
| Time (hr) | ARC1666 | ARC2982 | ARC2983 | ARC3901 |
| 0 | 1785.6 | 2048.9 | 1897.7 | 2129.6 |
| 8 | 547.7 | 1429.8 | 1885.6 | 2208.8 |
| % of Parent Aptamer | 30.7 | 69.8 | 99.4 | 103.7 |
| Remained | | | | |

All three of the new derivatives showed fewer or lower abundance of metabolites than ARC1666 in cynomolgus monkey serum. In particular, ARC3901 was stable in cynomolgus monkey serum at incubation times of 120 hours. As shown in FIGS. 7 and 8, site-specific modification of the ARC1666 sequence with phosphorothioate linkages effectively blocked putative nuclease-sensitive sites and dramatically enhanced the serum stability of the aptamer.

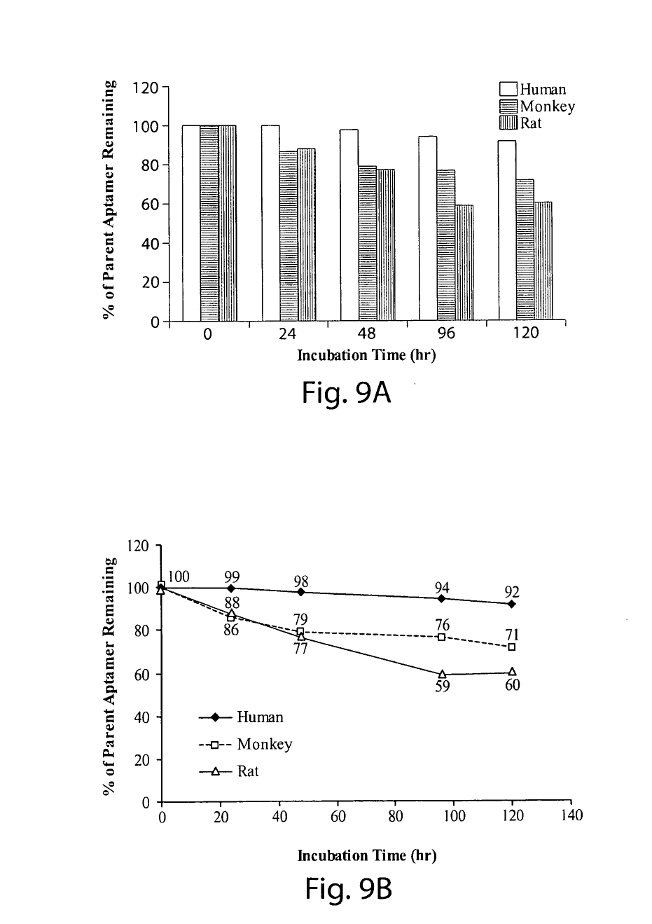

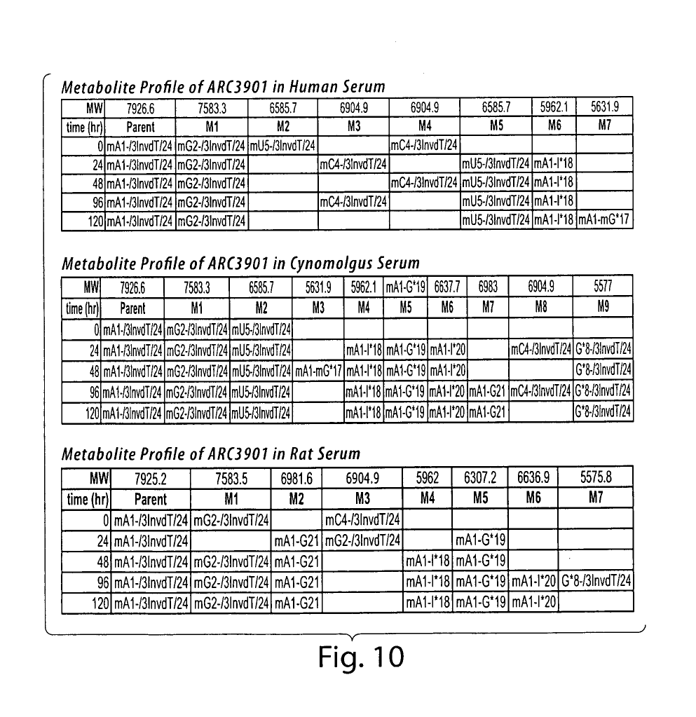

FIGS. 9A and 9B depict the percentage of intact ARC3901 remaining in human, rat and cyno serum after incubation for zero to 120 hours, as assessed by ESI-LC/MS. From this analysis, ARC3901 appears more stable in human than in monkey, and more stable in monkey than in rat. The metabolic profile of ARC3901, as assessed by ESI-LC/MS, in cyno, rat and human serum is given in the tables of FIG. 10.

Denaturing polyacrylamide gel electrophoresis (PAGE) may also be used to determine whether chemical modifications to the aptamer result in protection from nuclease cleavage. To prepare samples for analysis, 100 μM stock samples of ARC1666, 2982, 2983 and 3901 were diluted to a final concentration of 5 μM in 90% rat serum in a total volume of 100 μL. Each sample was mixed briefly and 20 μL aliquots were immediately removed from each sample into a 1.5 mL tube containing 100 μL of the stop buffer (80% formamide-5 mM EDTA) for “t=0” samples. These samples were frozen and stored at −20° C. until time of analysis. The remaining 80 μL of the serum-aptamer solution was incubated at 37° C. until the time of collection. After 24 hours, a 20 μL sample was removed and mixed with 100 μLs of stop buffer for “t=24” samples. Upon collection, the 24 hour time point sample was frozen and stored at −20° C. until time of analysis.

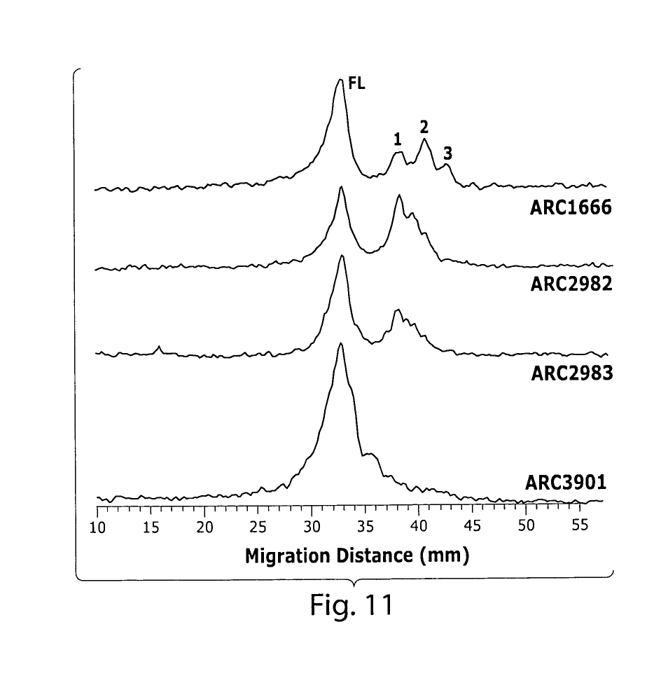

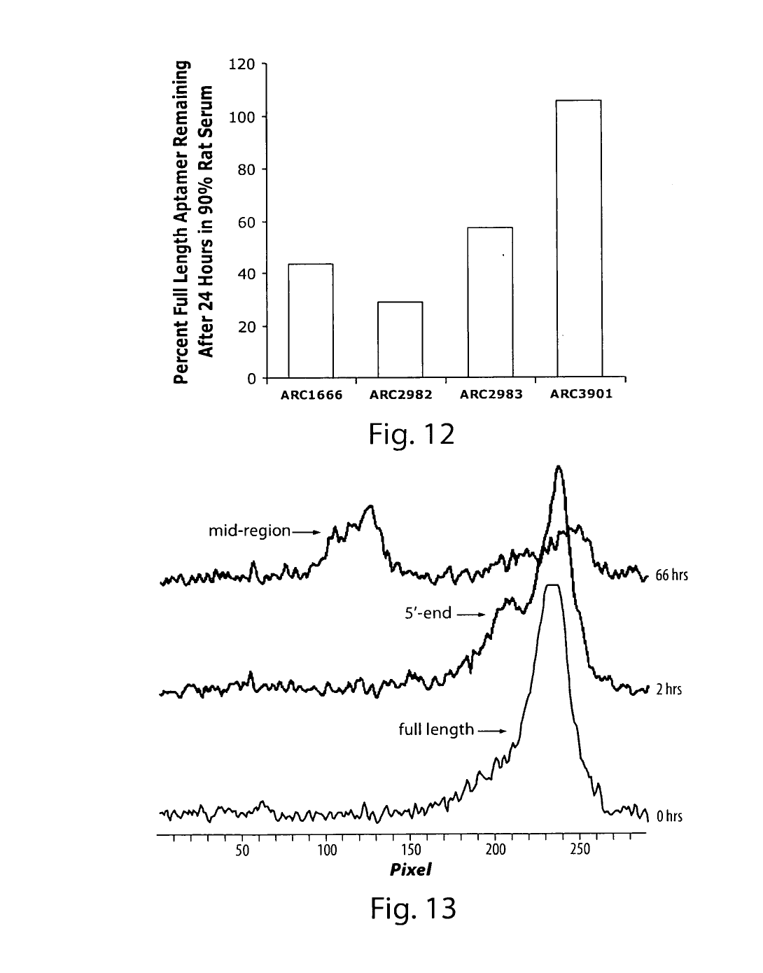

Disposable 1 mm plastic gel cassettes (Invitrogen, NC2010) were used to cast an 18% denaturing polyacrylamide gel by mixing 7.2 mL of SequaGel Concentrate (National Diagnostics, EC830), 1.8 mL of SequaGel Diluent (National Diagnostics, EC8430) and 1 mL of 10×TBE. Samples were prepared for electrophoresis by denaturing 150 ng of aptamer from each time point in the stop solution at 90° C. for 5 minutes. Samples were electrophoresed for 2 hours at 175 Volts and the gel was stained with a 1:5000 fold dilution of SYBR Gold Nucleic Acid Stain (Invitrogen S-11494) for 5 minutes. Bands on the PAGE gel corresponding to the full length aptamers and their metabolites were visualized with the Molecular Dynamics Storm Imager. FIG. 11 shows line graphs representing the relative intensity and migration distance of bands in the gel from the 24 hour time point for ARC 1666, ARC2982, ARC2983 and ARC3901. The peaks for the full length aptamer (FL) and the three metabolites (indicated by 1, 2 and 3) found in the ARC1666 sample are labeled. Modification of the ARC1666 aptamer resulted in the loss of metabolite peaks in the graphs for ARC2982 and ARC2983. Metabolites were absent after 24 hours in rat serum for ARC3901. The relative percentage of full length aptamer remaining after 24 hours in rat serum for ARC1666, ARC2982, ARC2983 and ARC3901 (FIG. 12) was calculated by integrating the area beneath the FL peak for the t=0 (data not shown) and t=24 time points.

A QSTAR ELITE ESI-LC/MS/MS method was also used for metabolite profiling to determine sites of aptamer cleavage. Prior to analysis with the QSTAR ELITE spectrometer (manufactured by Applied Biosystems of Foster City, Calif.), serum samples were prepared according to the Proteinase K sample assay set-up detailed above. In contrast to the ESI-LC/MS/MS method, however, serum samples analyzed according to the QSTAR ELITE LC/MS/MS method used reversed phase column and ion-pair reagents. HPLC separation was carried out on a Varian PLRP-s reversed phase polymer column (100 Å, 50×2.1 mm) as manufactured by Varian, Inc., of Palo Alto, Calif. The aptamers were eluted using ion-pair reagent TEA or DIPEA (5-10 mM), and HFIP (100-200 mM) in both water and MeOH mobile phases (pH=8.0). UV detection at 260 nm was captured with a diode array detector. Mass spectrometry detection was carried out with AB/Sciex QStar Elite hybrid LC/MS/MS Q-TOF mass spectrometer equipped with a TurboIonSpray as the LC/MS interface. ESI mass spectra were acquired in negative ion mode. Experimental masses that match expected cleavage masses were automatically reported as putative metabolites. The disappearance of integrated parent mass intensity can also be used to follow degradation of aptamers over time. The QSTAR ELITE LC/MS/MS method detects and quantifies the mass of aptamer metabolites with excellent resolution, sensitivity and accuracy. This method has been used to identify metabolites of aptamers in vitro in rat, monkey and human serum.

Once the metabolic stability of an aptamer has been optimized, the functionality of that aptamer may be further optimized, for example, by conjugation to a PEG moiety. It is not intended that the metabolically optimized aptamers described by embodiments of the present invention be limited to any specific molecular weight PEG or configuration of PEG. In one embodiment, a 20 kDa PEG was conjugated to the 3′ end of a metabolically optimized aptamer. In another embodiment, a 20 kDa PEG was conjugated to both the 3′ and 5′ ends of a metabolically optimized aptamer.

### Example 4

### Optimization of ARCXXXA for Nuclease Stability

The aptamers in this Example 4 have been blinded and are identified as ARCXXXA, ARCXXXB, ARCXXXC, etc. The aptamers in this Example 4 are different than the aptamers in Examples 5, 6 and 7, even though some of the aptamers are labeled the same. For the sake of clarity, there is no relation between the aptamers in this Example 4 and the aptamers in Examples 5, 6 and 7 (except that ARCXXXK in Example 4 is the same aptamer as ARCXXXA in Example 6).

Aptamer Optimization—PAGE and ELISA

ARCXXXK is a minimized, chemically modified aptamer that binds to a human target with high affinity. ARCXXXK was identified through a systematic approach involving multiple phases of aptamer synthesis, purification and assaying for functional activity and nuclease stability. Regions of nuclease cleavage in the parent aptamer, ARCXXXA, were identified by both LC/MS and PAGE analysis of aptamer samples digested in serum. Replacement of phosphodiester linkages with phosphorothioate linkages at sites of nuclease cleavage was the approach used to achieve a significant increase in plasma nuclease resistance and overall stability. The use of PAGE to identify regions of cleavage in ARCXXXA and use of a competitive ELISA assay to assess function of aptamer derivatives is described below.

Identification of Nuclease Sensitive Sites

5 μM aptamer plus a trace amount of 3′-³²P end labeled ARCXXXA was incubated in 90% mouse serum at 37° C. At each time point indicated in FIG. 13, 10 μL was withdrawn from each mixture and added to 100 μL stop mix (90% formamide, 10% 1×TBE, 0.1% SDS, Bromophenyl blue). 20 μL from each resulting sample was heated at 90° C. for 15 minutes, and 15 μL of the heated samples was loaded on a 15% denaturing PAGE gel, which was electrophoresed for 2 hours at 65 Watts. The gel was then exposed to a phosphor screen for 2 hours and the phosphor screen was scanned with a Molecular. Dynamics phosphorimager. The data was then analyzed with Molecular Dynamics Image Quant software. FIG. 13 shows serum analysis of ARCXXXA at 0 (solid line), 2 (large dashed line) and 66 (small dashed line) hours. The starting material peak is at ˜60 pixels on the X axis. The extra peak near 90 pixels on the X axis indicates a cleavage near the 5′-end of the molecule. The peak at ˜180 pixels indicates cleavage near the middle of the molecule. Therefore, the banding pattern in FIG. 13 indicated rapid cleavage near the 5′-end of the aptamer and accumulation of products resulting from cleavage near the middle of the aptamer.

ARCXXXA derivatives containing phosphorothioate substitutions near the 5′ end (ARCXXXB-ARCXXXD) were tested for activity in a competitive ELISA (Table 4) and stability in serum. All aptamers had similar activity, but only ARCXXXC had increased stability in serum (Table 5). ARCXXXC derivatives containing phosphorothioate substitutions near the middle of the aptamer (ARCXXXE-ARCXXXK) were designed to block cleavages in the middle of the oligonucleotide. ARCXXXK blocks the cleavage resulting in more stable aptamer and retains activity similar to ARCXXXC in the competitive ELISA (Table 4).

ELISA

To test the function of the optimized aptamers, an ELISA assay was set up to measure interference of target binding to its receptor. To capture receptor, 150 ng of receptor-Fc (R&D systems) in 100 μL of PBS (pH 7.4) was put onto a 96-well Maxisorb plate (NUNC #446612) and incubated overnight at 4° C. During the capture, 50 μL of various concentrations of synthetic RNA were mixed with 50 μL of 3.6 nM target (200 ng/mL) (R&D systems) (in PBS with 0.2% BSA) with a final target concentration at 1.8 nM (100 ng/mL) in PBS with 0.1% BSA, and incubated at room temperature for 1 hour. The capture solution was removed after an overnight incubation and the plate was washed three times with 200 μL of TBST (25 mM Tris-HCl pH 7.5, 150 mM NaCl and 0.01% Tween 20). The plate was then blocked with 200 μL TBST containing 5% nonfat dry milk for 30 minutes at room temperature. After blocking, the plate was washed with 200 μL of TBST three times at room temperature, and synthetic RNA:target mixture was added to the plate and incubated at room temperature for 1 hour. The plate was then washed with 200 μL of TBST three times, 100 μL of biotinylated goat anti-target antibody (1:1000; R&D Systems) was added and incubated for 1 hour at room temperature. After three washes with 200 μL of TBST, 100 μL of HRP linked Streptavidin (1:200; R&D systems #DY998) was added and incubated at room temperature for 0.5 hours. Then, the plate was washed again with 200 μL of TBST three times, 100 μL of TMP solution (Pierce, #34028) was added and incubated in the dark at room temp for 5 minutes. A solution of 100 μL containing 2 N H2SO4 was added to stop the reaction and the plate was read by SpectroMax at 450 nm.

TABLE 4
Aptamer Sequence and Activity in the Target: Receptor
Competitive ELISA

| ARC Number | ELISA IC₅₀ (nM) |
|---|---|
| XXXA | 3 |
| XXXB | 5 |
| XXXC | 5 |
| XXXD | 5 |
| XXXE | 15 |
| XXXF | 100 |
| XXXG | 20 |
| XXXH | 6 |
| XXXI | 6 |
| XXXJ | 5 |
| XXXK | 8 |

TABLE 5
Percent Aptamer Remaining After 96 Hours in Mouse Serum as Measured
by HPLC Using Standard Methods

| | % remaining after |
|---|---|
| | 96 hr in mouse |
| ARC# | serum |
| ARCXXXA | 16% |
| ARCXXXC | 59% |
| ARCXXXK | 77% |

### Example 5

### In Vitro Metabolic Profiling of ARCXXXA

The aptamers in this Example 5 have been blinded and are identified as ARCXXXA, ARCXXXB, ARCXXXC, etc. The aptamers in this Example 5 are different than the aptamers in Examples 4, 6 and 7, even though some of the aptamers are labeled the same. For the sake of clarity, there is no relation between the aptamers in this Example 5 and the aptamers in Examples 4, 6 and 7.

In vitro serum stability studies and metabolite profiling using HPLC and ESI-LC/MS methods (described above) were performed on lead compounds ARCXXXA, ARCXXXB and ARCXXXC. These lead compounds contained 2′-fluoropyrimidines and varying numbers of 2′-ribonucleotides and 2′OMe-ribonucleotides. It was anticipated that the relative metabolic stability of these aptamers might parallel the degree of 2′-OMe content: ARCXXXA<<<ARCXXXB<ARCXXXC, where ARCXXXC is the most fully stabilized by virtue of the least number of ribonucleotides and the most 2′-OMe modified nucleotides.

### In Vitro Stability Analysis

### HPLC and ESI LC/MS Analysis

The metabolic stability of aptamers ARCXXXA, ARCXXXB and ARCXXXC were evaluated in pooled Sprague-Dawley rat and human sera at concentrations of 10 μM and incubated at 37° C. for 0, 8, 24 and 120 hours. At each incubation time point, aliquots were withdrawn, flash-frozen in liquid nitrogen and stored at −80° C. until extraction with Proteinase K. All samples were analyzed by HPLC for percent full-length aptamer, as well as by ESI-LC/MS for metabolite profiling.

100 μM stocks of HPLC-purified ARCXXXA, ARCXXXB and ARCXXXC were prepared in water. Each construct was heated to 99° C. (92° C. measured) for 10 minutes in an Eppendorf Thermoshaker, chilled in an ice bath for 10 min, removed and left at room temperature until use.

The serum incubation assay for each species and concentration was assembled as shown below in Table 6. After assembly of each aptamer in rat or human serum (1.0 mL total incubation volume each), the solution was briefly mixed. 200 μL aliquots for each species and aptamer were immediately distributed into 1.5 mL tubes and flash-frozen in liquid nitrogen prior to storage at −80° C. for “t=0” samples. From the remaining 800 μL of serum solution, 200 μL of solution was added to fresh tubes labeled by aptamer number (ARCXXXA, ARCXXXB or ARCXXXC), time point (8, 24, 48 or 120 hrs) and serum species (rat or human), and all were placed in a 37° C. water bath with mild shaking until collection. All time points were immediately flash-frozen in liquid nitrogen and stored at −80° C. until time of analysis.

TABLE 6
Assay Setup

| Reagent | Volume (uL) |
|---|---|
| 10X ARCXXXX | 100 |
| Rat or Human Serum | 900 |

Prior to HPLC and ESI-LC/MS analysis, each sample was incubated with 100 μL of Proteinase K Buffer, and 100 μL of Proteinase K (3 mg/mL in Diluent Buffer) was added to each tube containing 200 μL of 10 μM ARCXXXA, ARCXXXB or ARCXXXC that had been incubated in rat or human serum for a specified period of time at 37° C. Each proteinase digestion reaction was incubated overnight at 55° C. with shaking. After incubation, proteinase digestion reaction tubes were centrifuged for 12 minutes at 14,000 rpm and 4° C. Approximately 350 μL of supernatant was collected from each tube and analyzed via HPLC and ESI-LC/MS. Briefly, the SAX-HPLC method may be described as follows:

Column/Medium: DNAPacPA-100

Platform/Instrument: Agilent 1100

Analysis Temperature (C): 80°

Analyzed Sample Volume (μL): 25

Solvent A: 25 mM Na Phosphate, pH 7.0, 25% ACN

Solvent B: 25 mM Na Phosphate, pH 7.0, 25% ACN, 400 mM NaClO₄

Gradient min (% B): 10

Gradient max (% B): 65

Gradient Duration (min): 22 minutes

Percent full length aptamer was calculated from the ratio of integrated chromatographic peak area of a given aptamer at each time point to that of its corresponding control (t=0) sample.

Samples were also subjected to ESI-LC/MS for metabolite profiling in order to determine sites of aptamer cleavage.

### In Vitro Stability Analysis

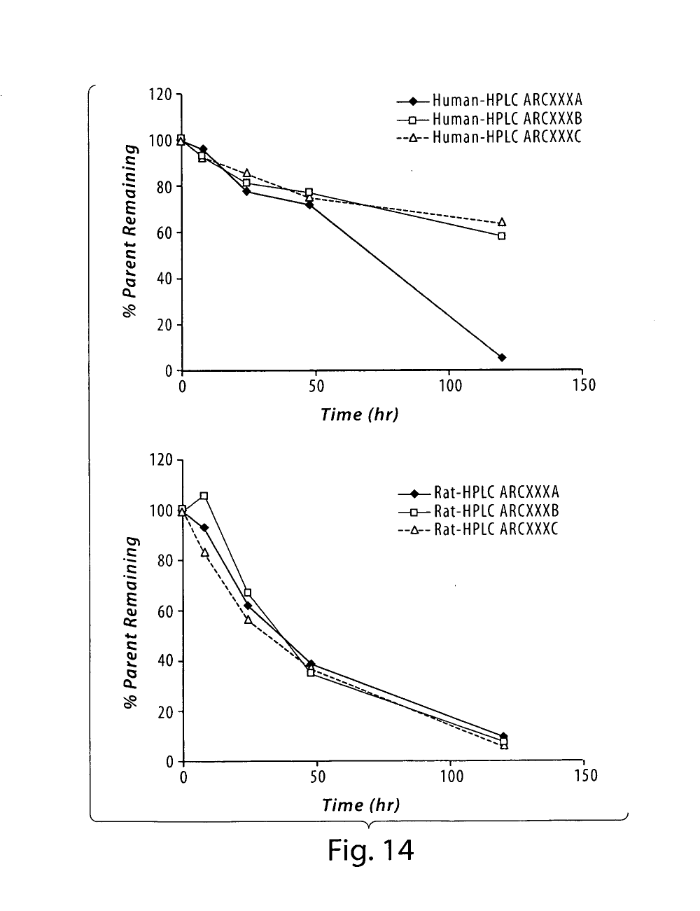

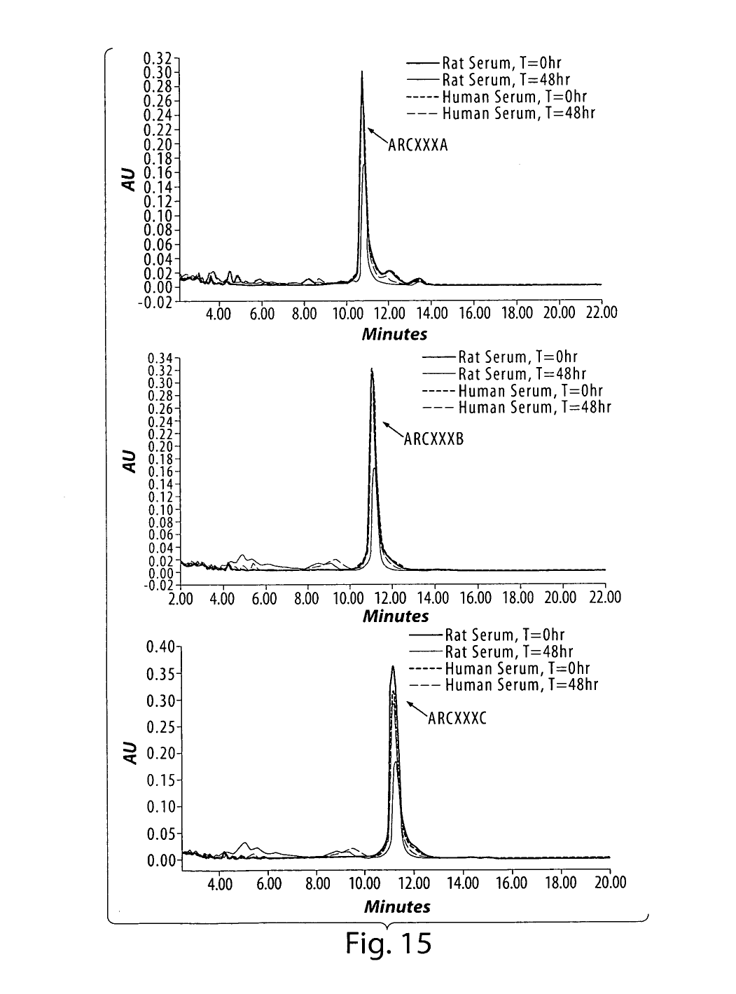

FIG. 14 graphically depicts the percent full length aptamer (round 1 optimized derivatives) remaining as a function of time for human and rat sera, based upon SAX-HPLC analyses. Detailed SAX-HPLC chromatograms of the aptamers following 48 hour incubation in rat and human serum are presented in FIG. 15. Percentages of full-length ARCXXXA, ARCXXXB and ARCXXXC remaining data for each serum species are reported below in Table 7. Following 24-hr incubations in pooled rat serum, approximately 62%, 67% and 57% of full-length ARCXXXA, ARCXXXB and ARCXXXC remained, respectively. Similarly, for 24 and 48-hr incubations in human serum, approximately 77% and 72%, 81% and 77%, and 85 and 75% of full-length ARCXXXA, ARCXXXB and ARCXXXC remained, respectively. The ARCXXXB sequence was most stable in rat serum and the ARCXXXC sequence was most stable in human serum.

TABLE 7
Percent Full-Length Round 1 Optimized Derivatives Remaining in Rat
and Human Serum

| | | | |
|---|---|---|---|
| Time | | | |
| (hr) | ARCXXXA | ARCXXXB | ARCXXXC |

Metabolic Serum Stability in Rat-HPLC

| | | | |
|---|---|---|---|
| 0 | 100 | 100 | 100 |
| 8 | 93 | 106 | 84 |
| 24 | 62 | 67 | 57 |
| 48 | 39 | 35 | 37 |
| 120 | 9 | 7 | 6 |

Metabolic Serum Stability in Human-HPLC

| | | | |
|---|---|---|---|
| 0 | 100 | 100 | 100 |
| 8 | 96 | 92 | 93 |
| 24 | 77 | 81 | 85 |
| 48 | 72 | 77 | 75 |
| 120 | 5 | 57 | 64 |

Examination of the ESI-LC/MS analyses from rat and human serum incubations of ARCXXXA, ARCXXXB and ARCXXXC indicated that the several major and minor cleavage sites are located in approximately the central third section of the sequences for all three aptamers (described in more detail below). The locations of these cleavage sites in the interior (middle) of the sequences suggest that endonucleases are largely responsible for the metabolism of these aptamers. Capping or modification of the 3′ and 5′ termini by inverted deoxythymidine and amino groups, respectively, likely act to stabilize the termini of the molecules against metabolic degradation.

ARCXXXA, a 38-mer with a 3′ inverted dT cap, was digested in either human or rat serum for 120 hours. Major and minor cleavage sites were determined by ESI-LC/MS. Major cleavage sites in human serum were between nucleotides 15 and 16, 22 and 23, and 23 and 24. Minor cleavage sites in human serum were between nucleotides 6 and 7, 12 and 13, 16 and 17, 17 and 18, 19 and 20, 20 and 21, and 21 and 22. Major cleavage sites in rat serum were between nucleotides 14 and 15, 15 and 16, 21 and 22, 22 and 23, and 23 and 24. Minor cleavage sites in rat serum were between nucleotides 9 and 10, 10 and 11, 11 and 12, 16 and 17, 18 and 19, 20 and 21, 24 and 25, and 25 and 26.

ARCXXXB, a 38-mer with a 3′ inverted dT cap, was digested in either human or rat serum for 120 hours. Major and minor cleavage sites were determined by ESI-LC/MS. Major cleavage sites in human serum were between nucleotides 20 and 21, 22 and 23, and 23 and 24. Minor cleavage sites in human serum were between nucleotides 10 and 11, 13 and 14, 17 and 18, 21 and 22, 24 and 25, and 25 and 26. Major cleavage sites in rat serum were between nucleotides 23 and 24. Minor cleavage sites in rat serum were between nucleotides 9 and 10, 10 and 11, 11 and 12, 12 and 13, 13 and 14, 14 and 15, 17 and 18, 20 and 21, 22 and 23, 24 and 25, 25 and 26, and 26 and 27.

ARCXXXC, a 38-mer with a 3′ inverted dT cap, was digested in either human or rat serum for 120 hours. Major and minor cleavage sites were determined by ESI-LC/MS. Major cleavage sites in human serum were between nucleotides 23 and 24, 24 and 25, and 25 and 26. Minor cleavage sites in human serum were between nucleotides 5 and 6, 17 and 18, 19 and 20, 20 and 21, 21 and 22, and 22 and 23. Major cleavage sites in rat serum were between nucleotides 23 and 24, 24 and 25, and 25 and 26. Minor cleavage sites in rat serum were between nucleotides 9 and 10, 10 and 11, 11 and 12, 12 and 13, 13 and 14, 20 and 21, 21 and 22, and 22 and 23.

Mapping of the cleavage sites of these aptamers would allow the design of more stable, optimized derivatives. This procedure of in vitro serum stability studies followed by metabolite profiling using HPLC and ESI-LC/MS methods can be used in an iterative fashion to design successively more stable, more fully optimized derivatives within just a few rounds of optimization.

### Example 6

### In Vitro Metabolic Profiling of ARCXXXA and its Derivatives

The aptamers in this Example 6 have been blinded and are identified as ARCXXXA, ARCXXXB, ARCXXXC, etc. The aptamers in this Example 6 are different than the aptamers in Examples 4, 5 and 7, even though some of the aptamers are labeled the same. For the sake of clarity, there is no relation between the aptamers in this Example 6 and the aptamers in Examples 4, 5 and 7 (except that ARCXXXK in Example 4 is the same aptamer as ARCXXXA in Example 6).

In vitro serum stability studies and metabolite profiling using HPLC and ESI-LC/MS methods (described above) were performed on ARCXXXA (which is the same aptamer as ARCXXXK in Example 4 and made according to the methods described in Example 4). Following initial metabolite profiling analyses on ARCXXXA, modifications were introduced to the aptamer with the goal of increasing the in vitro serum stability of the aptamer which generated aptamers ARCXXXC and ARCXXXD. ARCXXXB, which is the same as ARCXXXA but is capped at the 3′- and 5′- ends with NH2, was also synthesized. These derivatives contained additional phosphorothioate linkages at positions determined to be nuclease sensitive. By subjecting these aptamers to in vitro stability analysis and metabolite profiling, it was revealed that ARCXXXA derivatives with phosphorothioate modifications at sites of nuclease sensitivity had enhanced stability vs. ARCXXXA in rat and human sera.

### In Vitro Stability Analysis

### HPLC and ESI LC/MS Analysis

The metabolic stability of aptamers ARCXXXA, ARCXXXB, ARCXXXC and ARCXXXD were evaluated in pooled Sprague-Dawley rat and human sera at concentrations of 10 μM and incubated at 37° C. for 0, 8, 24 and 120 hours. At each incubation time point, aliquots were withdrawn, flash-frozen in liquid nitrogen and stored at −80° C. until extraction with Proteinase K. All samples were analyzed by HPLC for percent full-length aptamer, as well as by ESI-LC/MS/MS for metabolite profiling.

100 μM stocks of HPLC-purified ARCXXXA, ARCXXXB, ARCXXXC and ARCXXXD were prepared in water. Each construct was heated to 99° C. (92° C. measured) for 10 minutes in an Eppendorf Thermoshaker, chilled in an ice bath for 10 min, removed and left at room temperature until use.

The serum incubation assay for each species and concentration was assembled as shown below in Table 8. After assembly of each aptamer in rat or human serum (1.0 mL total incubation volume each), the solution was briefly mixed. 200 μL aliquots for each species and aptamer were immediately distributed into 1.5 mL tubes and flash-frozen in liquid nitrogen prior to storage at −80° C. for “t=0” samples. From the remaining 800 μL of serum solution, 200 μL of solution was added to fresh tubes labeled by aptamer number (ARCXXXA, ARCXXXB, ARCXXXC or ARCXXXD), time point (8, 24, 120 or 121 hrs) and serum species (rat or human), and all were placed in a 37° C. water bath with mild shaking until collection. All time points were immediately flash-frozen in liquid nitrogen and stored at −80° C. until time of analysis.

TABLE 8
Assay Setup

| Reagent | Volume (uL) |
|---|---|
| 10X ARCXXXX | 100 |
| rat or human serum | 900 |

Prior to HPLC and ESI-LC/MS analysis, each sample was incubated with 100 μL of Proteinase K Buffer, and 100 μL of Proteinase K (3 mg/mL in Diluent Buffer) was added to each tube containing 200 μL of 10 μM ARCXXXA, ARCXXXB, ARCXXXC or ARCXXXD that had been incubated in rat or human serum for a specified period of time at 37° C. Each proteinase digestion reaction was incubated overnight at 55° C. with shaking. After incubation, proteinase digestion reaction tubes were centrifuged for 12 minutes at 14,000 rpm and 4° C. Approximately 350 μL of supernatant was collected from each tube and analyzed via HPLC and ESI-LC/MS. Briefly, the SAX-HPLC method may be described as follows:

Column/Medium: DNAPacPA-100

Platform/Instrument: Agilent 1100

Analysis Temperature (C): 80°

Analyzed Sample Volume (μL): 25

Solvent A: 25 mM Na Phosphate, pH 7.0, 25% ACN

Solvent B: 25 mM Na Phosphate, pH 7.0, 25% ACN, 400 mM NaClO₄

Gradient min (% B): 10

Gradient max (% B): 65

Gradient Duration (min): 22 minutes

Percent full length aptamer was calculated from the ratio of integrated chromatographic peak area of a given aptamer at each time point to that of its corresponding control (t=0) sample.

Samples were also subjected to ESI-LC/MS for metabolite profiling in order to determine sites of aptamer cleavage.

### In Vitro Stability Analysis and Optimization

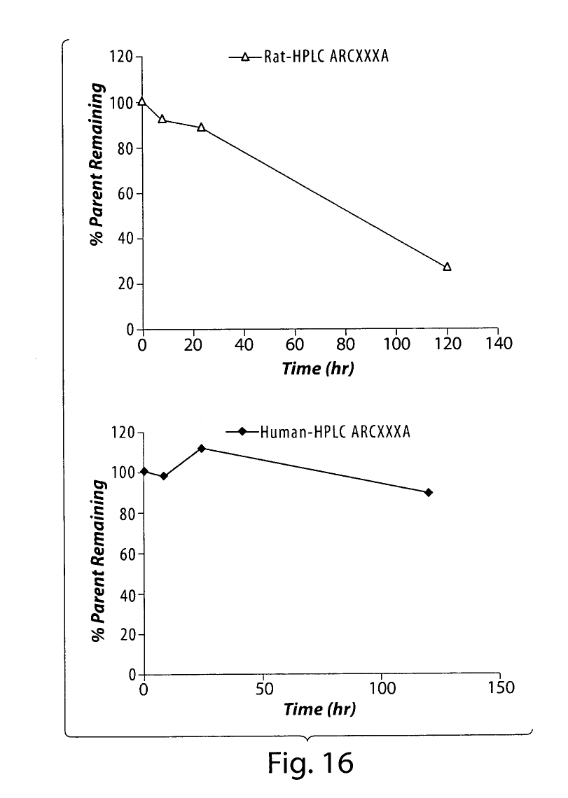

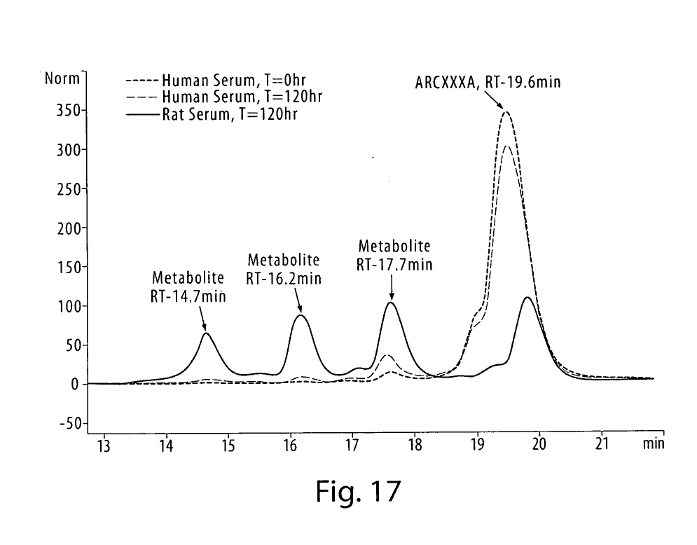

FIG. 16 graphically depicts ARCXXXA percent full length aptamer (round 1 optimized derivatives) remaining as a function of time for rat and human sera, based upon SAX-HPLC analyses. A detailed SAX-HPLC chromatogram of the ARCXXXA aptamer following 120 hour incubation in human and rat serum is presented in FIG. 17. Percentages of full-length remaining for ARCXXXA for each serum species is reported below in Table 9. Following 24-hr incubations in pooled rat serum, greater than 89% of full-length ARCXXXA remained. Similarly, for 24 and 120-hr incubations in human serum, greater than 90% and 100% of full length ARCXXXA remained, respectively.

TABLE 9
Percent Full-Length Original Aptamer Remaining in Rat
and Human Serum

| | |
|---|---|
| Time (hr) | ARCXXXA |

Metabolic Serum Stability in Rat-HPLC

| | |
|---|---|
| 0 | 100 |
| 8 | 92 |
| 24 | 89 |
| 120 | 26 |

Metabolic Serum Stability in Human-HPLC

| | |
|---|---|
| 0 | 100 |
| 8 | 98 |
| 24 | 112 |
| 120 | 90 |

Examination of the ESI-LC/MS analyses from human serum incubations of ARCXXXA at 48 hrs indicated that the primary cleavage site was 1 nucleotide in from the unprotected 5′ terminus (between nucleotides 1 and 2).

### In Vitro Stability Analysis and Optimization

Based upon the cleavage sites identified in ARCXXXA, two further, presumably more stable derivatives, were designed. These derivatives of ARCXXXA have additional phosphorothioate linkages at nuclease-sensitive sites. Additionally, a control molecule, ARCXXXB, which is the same as ARCXXXA but is capped at the 3′- and 5′- ends with NH2, was also synthesized so that it could be compared directly to ARCXXXC and ARCXXXD, which also contain terminal NH2 caps.

These three aptamers were subjected to in vitro serum stability in rat and human sera using HPLC and ESI-LC/MS/MS methods like those described above.

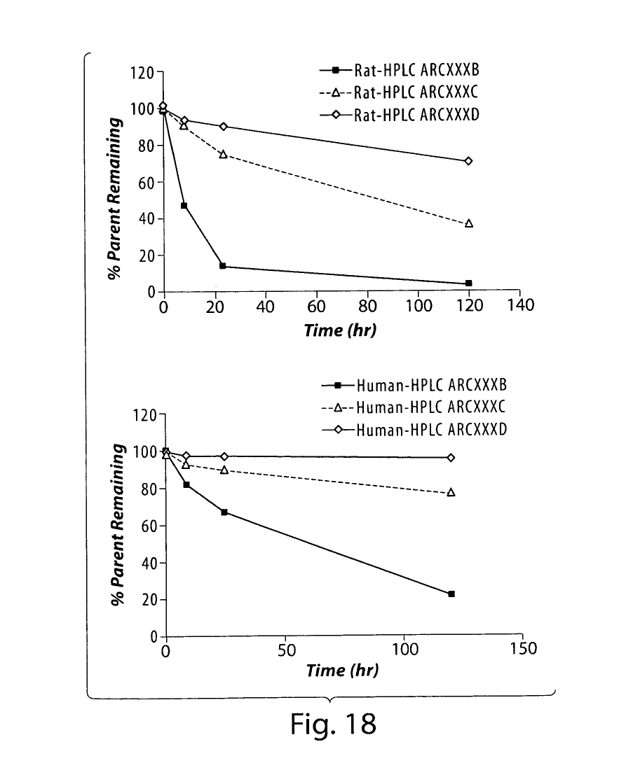

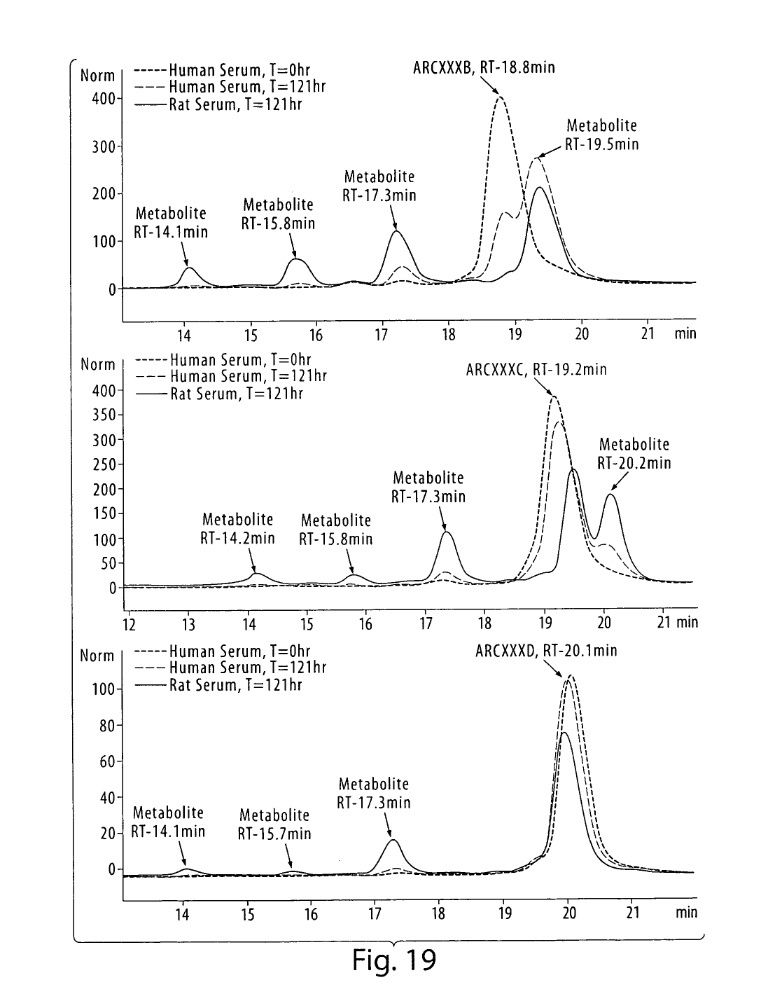

FIG. 18 graphically depicts ARCXXXB, ARCXXXC and ARCXXXD percent full length aptamer (round 2 optimized derivatives) remaining as a function of time for human and rat sera, based upon SAX-HPLC analyses. Detailed SAX-HPLC chromatograms of the ARCXXXB, ARCXXXC and ARCXXXD aptamers (round 3 optimized derivatives) following 121 hour incubation in rat and human serum are presented in FIG. 19. Percentages of full-length remaining for ARCXXXB, ARXXXXC and ARCXXXD for each serum species are reported below in Table 10. Following 24-hr incubations in pooled rat serum, greater than 90% of full-length ARCXXXD remained. Similarly, for 24 and 120-hr incubations in human serum, greater than approximately 97% and 96% of full length ARCXXXD remained. One of the two round 2 derivatives has greater metabolic stability than the original lead compound, ARCXXXA.

TABLE 10
Percent Full-Length of Round 2 Optimized Derivatives Remaining in
Rat and Human Serum

| | | | | |
|---|---|---|---|---|
| Time | | | | |
| (hr) | ARCXXXA | ARCXXXB | ARCXXXC | ARCXXXD |

Metabolic Serum Stability in Rat-HPLC

| | | | | |
|---|---|---|---|---|
| 0 | 100 | 100 | 100 | 100 |
| 8 | 92 | 46 | 90 | 94 |
| 24 | 89 | 13 | 74 | 90 |
| 120 | 26 | 3 | 37 | 70 |

Metabolic Serum Stability in Human-HPLC

| | | | | |
|---|---|---|---|---|
| 0 | 100 | 100 | 100 | 100 |
| 8 | 98 | 82 | 93 | 98 |
| 24 | 112 | 67 | 89 | 97 |
| 120 | 90 | 22 | 77 | 96 |

Examination of the ESI-LC/MS analyses from rat and human serum incubations at 121 hours allowed deduction of the sites of cleavage, based upon the mass associated with the chromatogram peaks. Deduced sites of cleavage for these derivatives are provided below.

ARCXXXB, a 44-mer, was digested in either human or rat serum for 121 hours. Cleavage sites were determined by ESI-LC/MS. Cleavage sites in human serum were between nucleotides 1 and 2, 2 and 3, and 7 and 8. Cleavage sites in rat serum were between nucleotides 2 and 3, 7 and 8, 8 and 9, 13 and 14, 16 and 17, and 30 and 31.

ARCXXXC, a 44-mer, was digested in either human or rat serum for 121 hours. Cleavage sites were determined by ESI-LC/MS. Cleavage sites in human serum were between nucleotides 1 and 2. Cleavage sites in rat serum were between nucleotides 1 and 2, 7 and 8, 8 and 9, 13 and 14, and 16 and 17.

ARCXXXD, a 44-mer, was digested in either human or rat serum for 121 hours. Cleavage sites were determined by ESI-LC/MS. Cleavage sites in human serum were between nucleotides 7 and 8. Cleavage sites in rat serum were between nucleotides 7 and 8, 8 and 9, and 16 and 17.

The high resolution metabolite profiling using the ESI-LC/MS analysis indicates that the 2 derivatives show fewer metabolites than ARCXXXA, with one derivative, ARCXXXD, showing acceptable metabolic stability in rat (greater than 80% parent remaining at 24 hours) and human (greater than 80% parent remaining at 48 hours) serum.

Optimization round 1 resulted in improvements in the in vitro stability of at least one derivative compared to that of the original lead compound, ARCXXXA. From the round 1 derivatives, a single aptamer (ARCXXXD) was then selected as being the most metabolically stable and suitable for further therapeutic preclinical development.

### Example 7

### In Vitro Metabolic Profiling of ARCXXXA and its Derivatives

The aptamers in this Example 7 have been blinded and are identified as ARCXXXA, ARCXXXB, ARCXXXC, etc. The aptamers in this Example 7 are different than the aptamers in Examples 4, 5 and 6, even though some of the aptamers are labeled the same. For the sake of clarity, there is no relation between the aptamers in this Example 7 and the aptamers in Examples 4, 5 and 6.

In vitro serum stability studies and metabolite profiling using HPLC and ESI-LC/MS methods (described above) were performed on lead compound, ARCXXXA, which is derived from clone ARCXXXG (Table 11). Following initial metabolite profiling analyses on ARCXXXA, two rounds of optimization were performed with the goal of increasing the in vitro serum stability of the aptamer. A round of optimization is defined as the design and synthesis of substituted aptamer derivatives followed by in vitro serum stability studies, including ESI-LC/MS metabolite profiling and activity assays. In the first round of optimization, based upon the metabolite profile observed for ARCXXXA, five derivatives were designed and synthesized. These derivatives contained either phosphorothioate linkages or sequence variations at positions determined to be nuclease sensitive. These derivatives were subjected to in vitro stability analysis. In the second round of optimization, based upon the metabolite profiles of round 1 derivatives ARCXXXB, ARCXXXC, ARCXXXD, ARCXXXE and ARCXXXF, fourteen additional derivatives were designed, synthesized and tested. These derivatives contained either additional or different phosphorothioate linkages or sequence variations at positions determined to be nuclease sensitive in the previous round of optimization. After narrowing the round 2 derivatives on the basis of in vitro activity, seven derivatives were subjected to in vitro stability analysis and metabolite profiling. The most stable round 2 derivatives showed significantly greater in vitro stability than ARCXXXA in rat and human sera, and contain the same or fewer phosphorothioate substitutions.

TABLE 11
Original Aptamer Leads

| ARC No. | IC50 (nM) |
|---|---|
| ARCXXXG | 6 |
| ARCXXXA | 3 |

### In Vitro Stability Analysis

### HPLC and ESI LC/MS Analysis

The metabolic stability of aptamers ARCXXXA, ARCXXXB, ARCXXXC, ARCXXXD, ARCXXXE and ARCXXXF were evaluated in pooled Sprague-Dawley rat and human sera at concentrations of 10 μM and incubated at 37° C. for 0, 2, 8 and 48 hours. At each incubation time point, aliquots were withdrawn, flash-frozen in liquid nitrogen and stored at −80° C. until extraction with Proteinase K. All samples were analyzed by HPLC for percent full-length aptamer, as well as by ESI-LC/MS for metabolite profiling.

100 μM stocks of HPLC-purified ARCXXXA, ARCXXXB, ARCXXXC, ARCXXXD, ARCXXXE and ARCXXXF were prepared in water. Each construct was heated to 99° C. (92° C. measured) for 10 minutes in an Eppendorf Thermoshaker, chilled in an ice bath for 10 min, removed and left at room temperature until use.

The serum incubation assay for each species and concentration was assembled as shown below in Table 12. After assembly of each aptamer in rat or human serum (1.0 mL total incubation volume each), the solution was briefly mixed. 200 μL aliquots for each species and aptamer were immediately distributed into 1.5 mL tubes and flash-frozen in liquid nitrogen prior to storage at −80° C. for “t=0” samples. From the remaining 800 μL of serum solution, 200 μL of solution was added to fresh tubes labeled by aptamer number (ARCXXXA, ARCXXXB, ARCXXXC, ARCXXXD, ARCXXXE or ARCXXXF), time point (2, 8 or 48 hrs) and serum species (rat or human), and all were placed in a 37° C. water bath with mild shaking until collection. All time points were immediately flash-frozen in liquid nitrogen and stored at −80° C. until time of analysis.

TABLE 12
Assay Setup

| Reagent | Volume (uL) |
|---|---|
| 10X ARCXXXX | 100 |
| rat or Human Serum | 900 |

Prior to HPLC and ESI-LC/MS/MS analysis, each sample was incubated with 100 μL of Proteinase K Buffer, and 100 μL of Proteinase K (3 mg/mL in Diluent Buffer) was added to each tube containing 200 μL of 10 μM ARCXXXA, ARCXXXB, ARCXXXC, ARCXXXD, ARCXXXE or ARCXXXF that had been incubated in rat or human serum for a specified period of time at 37° C. Each proteinase digestion reaction was incubated overnight at 55° C. with shaking. After incubation, proteinase digestion reaction tubes were centrifuged for 12 minutes at 14,000 rpm and 4° C. Approximately 350 μL of supernatant was collected from each tube and analyzed via HPLC and ESI-LC/MS/MS. Briefly, the SAX-HPLC method may be described as follows:

Column/Medium: DNAPacPA-100

Platform/Instrument: Agilent 1100

Analysis Temperature (C): 80°

Analyzed Sample Volume (μL): 25

Solvent A: 25 mM Na Phosphate, pH 7.0, 25% ACN

Solvent B: 25 mM Na Phosphate, pH 7.0, 25% ACN, 400 mM NaClO₄

Gradient min (% B): 10

Gradient max (% B): 65

Gradient Duration (min): 22 minutes

Percent full length aptamer was calculated from the ratio of integrated chromatographic peak area of a given aptamer at each time point to that of its corresponding control (t=0) sample.

Samples were also subjected to ESI-LC/MS for metabolite profiling in order to determine sites of aptamer cleavage.

### Round 1 Optimization

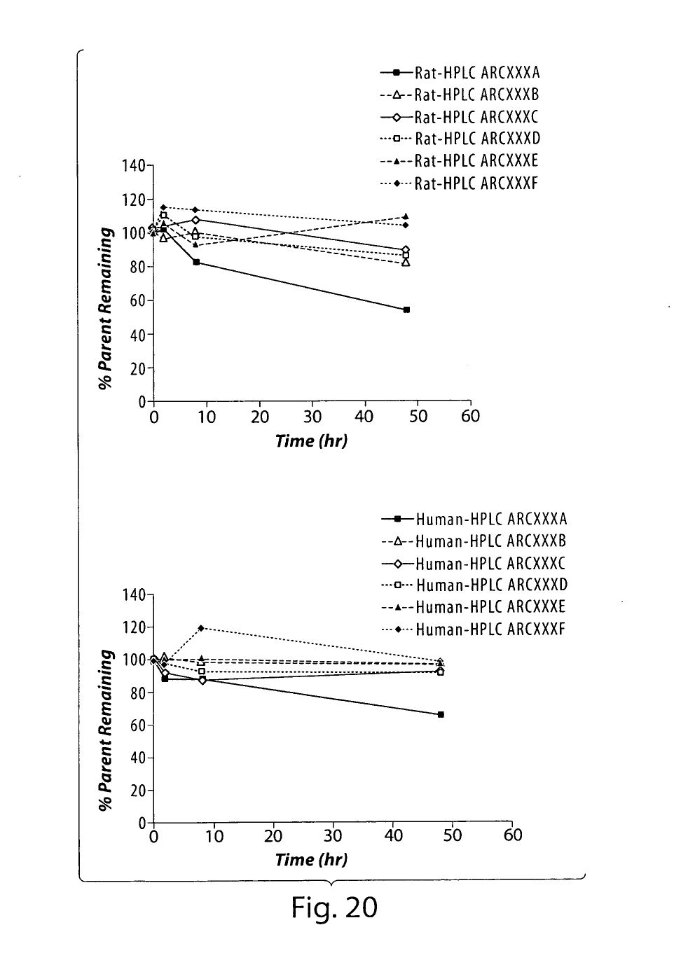

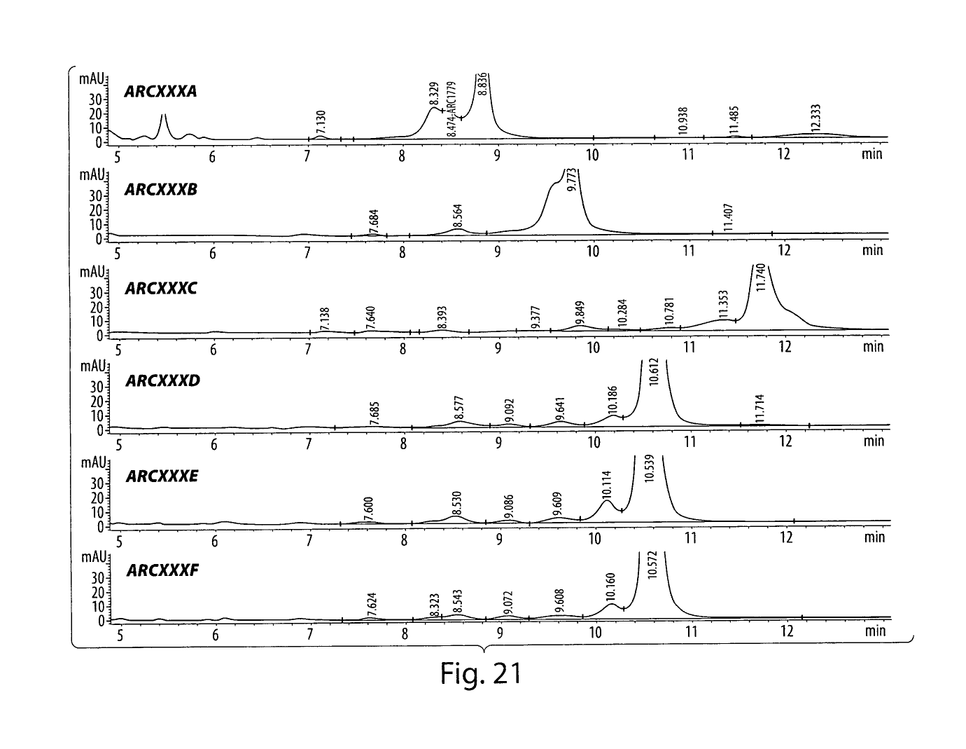

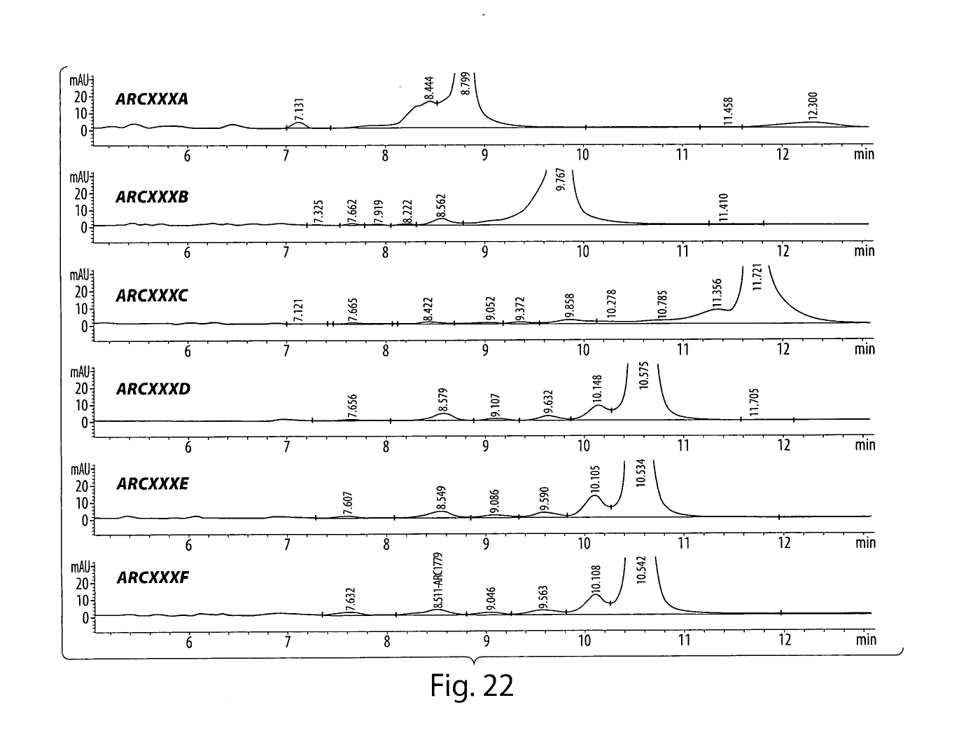

FIG. 20 graphically depicts the percent full length aptamer (round 1 optimized derivatives) remaining as a function of time for rat and human sera, based upon SAX-HPLC analyses. Detailed SAX-HPLC chromatograms of the aptamers following 48 hour incubation in rat and human serum are presented in FIGS. 21 and 22 respectively. Percentages of full-length ARCXXXA, ARCXXXB, ARCXXXC, ARCXXXD, ARCXXXE and ARCXXXF remaining for each serum species are reported below in Table 13. Following 48-hr incubations in pooled rat serum, greater than 81% of full-length ARCXXXB, ARCXXXC, ARCXXXD, ARCXXXE and ARCXXXF remained. Similarly, for 48-hr incubations in human serum, greater than 92% of full length ARCXXXB, ARCXXXC, ARCXXXD, ARCXXXE and ARCXXXF remained. The five ARCXAAA derivatives have significantly greater metabolic stability than their precursor, ARCXXXA.

TABLE 13
Percent Full-Length Round 1 Optimized Derivatives
Remaining in Rat and Human Serum

| | | | | | | |
|---|---|---|---|---|---|---|
| Time | | | | | | |
| (hr) | ARCXXXA | ARCXXXB | ARCXXXC | ARCXXXD | ARCXXXE | ARCXXXF |

Metabolic Serum Stability in Rat-HPLC

| | | | | | | |
|---|---|---|---|---|---|---|
| 0 | 100 | 100 | 100 | 100 | 100 | 100 |
| 2 | 102 | 96 | 104 | 110 | 104 | 115 |
| 8 | 82 | 100 | 107 | 98 | 92 | 113 |
| 48 | 54 | 81 | 89 | 86 | 108 | 104 |

Metabolic Serum Stability in Human-HPLC

| | | | | | | |
|---|---|---|---|---|---|---|
| 0 | 100 | 100 | 100 | 100 | 100 | 100 |
| 2 | 89 | 101 | 93 | 98 | 100 | 97 |
| 8 | 88 | 99 | 88 | 93 | 100 | 119 |
| 48 | 66 | 96 | 93 | 92 | 96 | 98 |

Examination of the ESI-LC/MS analyses from rat and human serum incubations at 48 hours allowed deduction of the sites of cleavage, based upon the mass associated with the chromatogram peaks. Deduced sites of cleavage for round 1 optimized derivatives are described below.

ARCXXXA, a 35-mer, was digested in either human or rat serum for 48 hours. Cleavage sites were determined by ESI-LC/MS. Cleavage sites in human serum were between nucleotides 11 and 12, 20 and 21, and 31 and 32. Cleavage sites in rat serum were between nucleotides 1 and 2, 11 and 12, 12 and 13, and 20 and 21.

ARCXXXB, a 35-mer, was digested in either human or rat serum for 48 hours. Cleavage sites were determined by ESI-LC/MS. Cleavage sites in human serum were between nucleotides 1 and 2, and 11 and 12. Cleavage sites in rat serum were between nucleotides 1 and 2, 8 and 9, and 11 and 12.

ARCXXXC, a 35-mer, was digested in either human or rat serum for 48 hours. Cleavage sites were determined by ESI-LC/MS. Cleavage sites in human serum were between nucleotides 7 and 8. No cleavage sites in rat serum were detected.

ARCXXXD, a 32-mer, was digested in either human or rat serum for 48 hours. Cleavage sites were determined by ESI-LC/MS. Cleavage sites in human serum were between nucleotides 4 and 5, and 28 and 29. Cleavage sites in rat serum were between nucleotides 1 and 2, 3 and 4, 4 and 5, and 28 and 29.

ARCXXXE, a 32-mer, was digested in either human or rat serum for 48 hours. Cleavage sites were determined by ESI-LC/MS. Cleavage sites in human serum were between nucleotides 1 and 2. Cleavage sites in rat serum were between nucleotides 1 and 2.

ARCXXXF, a 32-mer, was digested in either human or rat serum for 48 hours. Cleavage sites were determined by ESI-LC/MS. Cleavage sites in human serum were between nucleotides 1 and 2. Cleavage sites in rat serum were between nucleotides 1 and 2.

The high resolution metabolite profiling using the ESI-LC/MS analysis indicates that all five of the new derivatives show fewer metabolites than ARCXXXA and acceptable metabolic stability in rat (greater than 90% parent remaining at 24 hours) and human (greater than 90% parent remaining at 48 hours) serum.

The aptamers were also tested for activity in a cell-based activity assay. The results are summarized in Table 14.

TABLE 14
Activity of ARCXXXA Derivatives in a Cell-Based Assay

| ARC No. | IC50 (nM) |
|---|---|
| ARCXXXG | 6 |
| ARCXXXA | 3 |
| ARCXXXB | 3 |
| ARCXXXC | 5 |
| ARCXXXD | 33 |
| ARCXXXE | 30 |
| ARCXXXF | 37 |

ARCXXXB and ARCXXXC showed acceptable potency (IC₅₀≦10 nM) however, ARCXXXD, ARCXXXE and ARCXXXF did not.

ARCXXXB was improved with respect to in vitro stability and potency, had fewer compositional changes compared to ARCXXXA, and had the best activity of the new derivatives. ARCXXXC also showed improvement, but was slightly less active and represented the most substituted of the ARCXXXA derivatives (13 phosphorothioates compared to 7 for ARCXXXB). Subsequent investigations and optimization focused on alternative aptamer compositions that showed in vitro stability and potency while containing fewer phosphorothioate linkages than ARCXXXC.

### Round 2 Optimization

Based upon the data generated in round 1 optimization and on an evaluation of the previous data from the optimization of ARCXXXG, three strategies were used in designing panels of aptamers for round 2 optimization (Table 15). Panel I contains derivatives of ARCXXXC with fewer phosphorothioate linkages. Panel II contains derivatives of ARCXXXA with substitutions that block putative nuclease-sensitive sites with 2′-O-Me substitutions instead of phosphorothioates. ARCXXXH is a highly stable (but less potent) derivative of ARCXXXG. Panel III contains derivatives of ARCXXXH with 2′-O-Me substitutions at sites of metabolism in the context of ARCXXXA. None of the above three strategies is preferred relative to the others. Either strategy may be used or a combination of the strategies may be used.

Cell-based activity assays were performed on all fourteen derivatives from the round 2 derivatives of the three panels (Table 15).

TABLE 15
Optimization Round 2 Aptamers and Activity Results

| ARC No. | IC50 (nM) |
|---|---|
| ARCXXXI | 1 |
| ARCXXXJ | 1 |
| ARCXXXK | 5 |
| ARCXXXL | 5 |
| ARCXXXM | 16 |
| ARCXXXN | 12 |
| ARCXXXO | 7 |
| ARCXXXP | 11 |
| ARCXXXQ | 50 |
| ARCXXXR | 60 |
| ARCXXXS | 6 |
| ARCXXXT | 9 |
| ARCXXXU | 20 |
| ARCXXXV | 50 |

Of the fourteen round 2 derivatives, seven showed acceptable activity (Table 16). These seven aptamers, along with ARCXXXA, were subjected to in vitro serum stability in rat and human sera using HPLC and ESI-LC/MS methods like those described above for the first round of optimization.

TABLE 16
Optimization Aptamers with Acceptable Activity to be
Further Characterized

| ARC No. | IC50 (nM) |
|---|---|
| ARCXXXI | 1 |
| ARCXXXJ | 1 |
| ARCXXXK | 5 |
| ARCXXXL | 5 |
| ARCXXXO | 7 |
| ARCXXXS | 6 |
| ARCXXXT | 9 |

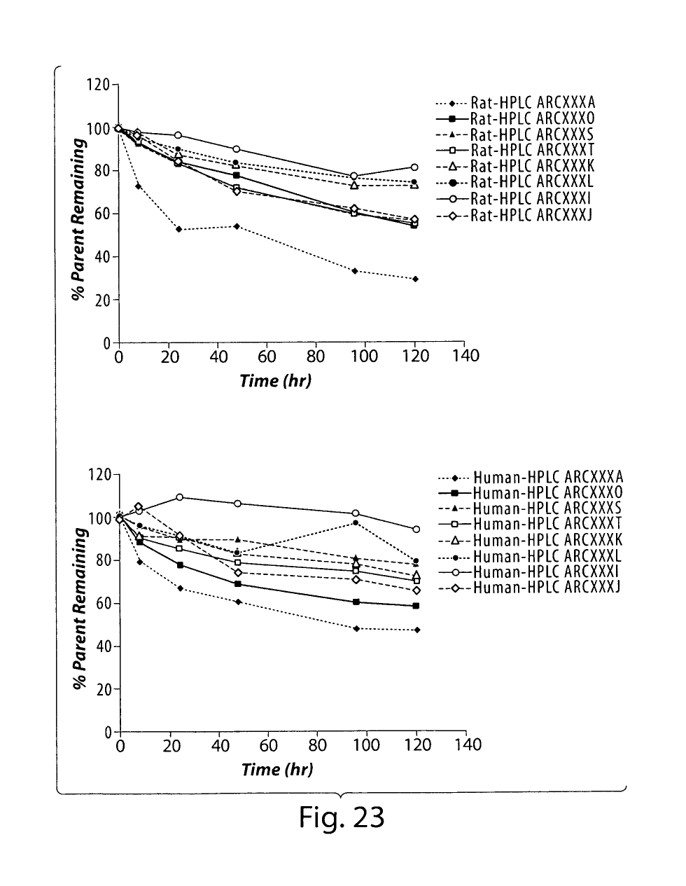

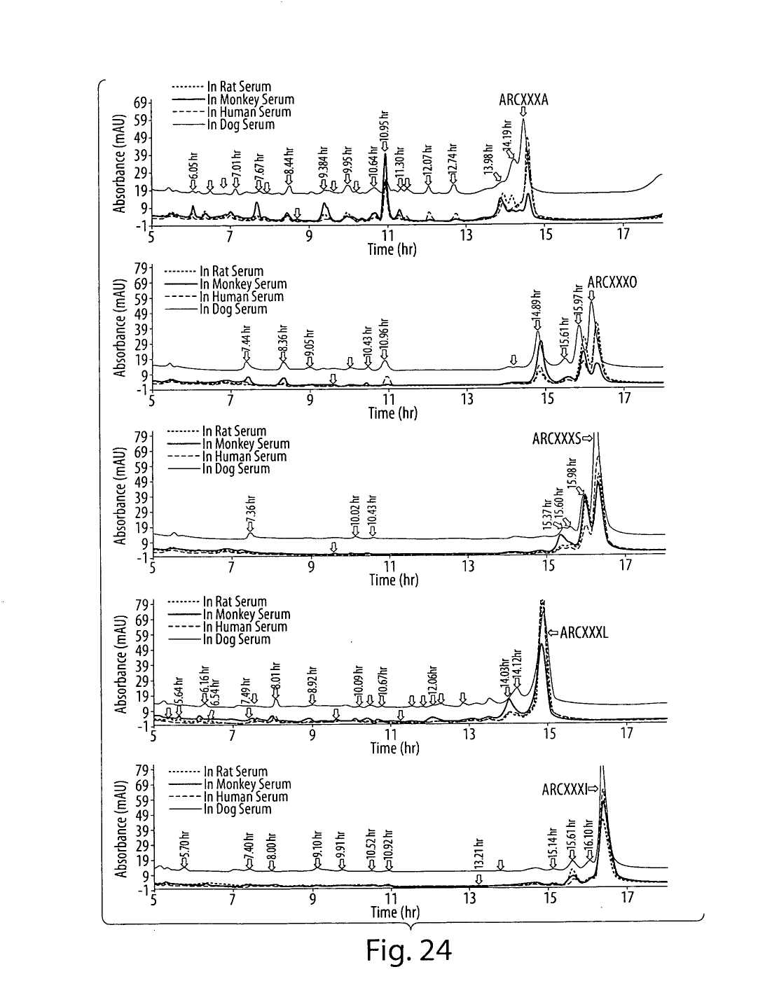

FIG. 23 graphically depicts the percent full length aptamer (round 2 optimized derivatives) remaining as a function of time for rat and human sera, based upon SAX-HPLC analyses. Detailed SAX-HPLC chromatograms of the aptamers following 48 hour incubation in rat, monkey, human and dog serum are presented in FIG. 24. Percentages of full-length ARCXXXA, ARCXXXB, ARCXXXC, ARCXXXD, ARCXXXE and ARCXXXF remaining for each serum species are reported below in Table 17. Following 48-hr incubations in pooled rat serum, greater than 82% of full-length ARCXXXK, ARCXXXL and ARCXXXI remained. Similarly, for 48-hr incubations in human serum, greater than approximately 80% of full length ARCXXXS, ARCXXXT, ARCXXXK, ARCXXXL and ARCXXXI remained. Five of the seven round 2 derivatives had significantly greater metabolic stability than the original lead compound, ARCXXXA.

TABLE 17
Percent Full-Length of Round 2 Optimized Derivatives Remaining in Rat and Human Serum

| | | | | | | | | |
|---|---|---|---|---|---|---|---|---|
| Time (hr) | ARCXXXA | ARCXXXO | ARCXXXS | ARCXXXT | ARCXXXK | ARCXXXL | ARCXXXI | ARCXXXJ |

Metabolic Serum Stability in Rat-HPLC

| | | | | | | | | |
|---|---|---|---|---|---|---|---|---|
| 0 | 100 | 100 | 100 | 100 | 100 | 100 | 100 | 100 |
| 8 | 73 | 92 | 93 | 94 | 95 | 93 | 97 | 97 |
| 24 | 53 | 83 | 83 | 83 | 87 | 90 | 96 | 84 |
| 48 | 54 | 77 | 72 | 72 | 82 | 83 | 90 | 70 |
| 96 | 33 | 60 | 60 | 60 | 72 | 76 | 77 | 62 |
| 120 | 29 | 54 | 57 | 56 | 72 | 74 | 81 | 55 |

Metabolic Serum Stability in Human-HPLC

| | | | | | | | | |
|---|---|---|---|---|---|---|---|---|
| 0 | 100 | 100 | 100 | 100 | 100 | 100 | 100 | 100 |
| 8 | 79 | 88 | 96 | 90 | 105 | 90 | 103 | 96 |
| 24 | 67 | 78 | 90 | 85 | 92 | 90 | 109 | 91 |
| 48 | 60 | 69 | 89 | 79 | 83 | 83 | 106 | 74 |
| 96 | 48 | 60 | 81 | 74 | 77 | 97 | 101 | 71 |
| 120 | 47 | 58 | 78 | 70 | 72 | 78 | 94 | 65 |

Examination of the ESI-LC/MS analyses from rat and human serum incubations at 48 hours allowed deduction of the sites of cleavage, based upon the mass associated with the chromatogram peaks. Deduced sites of cleavage for round 2 optimized derivatives are described below.

ARCXXXO, a 34 mer, was digested in either human or rat serum for 48 hours. Cleavage sites were determined by ESI-LC/MS. Cleavage sites in human serum were between nucleotides 11 and 12, and 12 and 13. Cleavage sites in rat serum were between nucleotides 3 and 4, 4 and 5, 5 and 6, and 19 and 20.

ARCXXXS, a 34-mer, was digested in either human or rat serum for 48 hours. Cleavage sites were determined by ESI-LC/MS. Cleavage sites in human serum were between nucleotides 2 and 3, and 11 and 12. Cleavage sites in rat serum were between nucleotides 3 and 4, and 4 and 5.

ARCXXXT, a 34-mer, was digested in either human or rat serum for 48 hours. Cleavage sites were determined by ESI-LC/MS. No cleavage sites in human serum were identified. Cleavage sites in rat serum were between nucleotides 3 and 4, 4 and 5, and 5 and 6.

ARCXXXK, a 34-mer, was digested in either human or rat serum for 48 hours. Cleavage sites were determined by ESI-LC/MS. Cleavage sites in human serum were between nucleotides 4 and 5, 31 and 32, and 32 and 33. Cleavage sites in rat serum were between nucleotides 3 and 4, 4 and 5, and 31 and 32.

ARCXXXL, a 34-mer, was digested in either human or rat serum for 48 hours. Cleavage sites were determined by ESI-LC/MS. Cleavage sites in human serum were between nucleotides 4 and 5, 31 and 32, and 32 and 33. Cleavage sites in rat serum were between nucleotides 3 and 4, 4 and 5, 5 and 6, and 31 and 32.

ARCXXXI, a 34-mer, was digested in either human or rat serum for 48 hours. Cleavage sites were determined by ESI-LC/MS. Cleavage sites in human serum were between nucleotides 7 and 8. Cleavage sites in rat serum were between nucleotides 3 and 4, and 7 and 8.

ARCXXXJ, a 34-mer, was digested in either human or rat serum for 48 hours. Cleavage sites were determined by ESI-LC/MS. Cleavage sites in human serum were between nucleotides 9 and 10. Cleavage sites in rat serum were between nucleotides 7 and 8, and 9 and 10.

The high resolution metabolite profiling using the ESI-LC/MS analysis indicated that all seven of the new derivatives (round 2 optimized derivatives) show fewer metabolites than ARCXXXA and acceptable metabolic stability in rat (greater than 80% parent remaining at 24 hours) and human (greater than 80% parent remaining at 48 hours) serum.

Optimization rounds 1 and 2 resulted in successive improvements in the in vitro stability of the derivatives from each round compared to that of the original lead compound, ARCXXXA. From the round 2 derivatives, a single aptamer (ARCXXXI) was then selected as being the most metabolically stable and suitable for further therapeutic preclinical development.

The invention having now been described by way of written description and example, those of skill in the art will recognize that the invention can be practiced in a variety of embodiments and that the description and examples above are for purposes of illustration and not limitation of the following claims.

What is claimed is:

1) A method for modifying an aptamer comprising the steps of:
a) incubating a parent aptamer with a test fluid to result in a mixture;
b) analyzing the mixture to identify metabolites of the parent aptamer, thereby detecting at least one aptamer cleavage site in the parent aptamer; and
c) introducing a chemical substitution at a position proximal to the at least one aptamer cleavage site to result in a modified aptamer.

2) The method of claim 1, wherein the test fluid is a biological matrix, a low pH fluid, a high pH fluid, a room temperature fluid or a body temperature fluid.

3) The method of claim 2, wherein the biological matrix is selected from the group consisting of one or more of: serum; plasma; cerebral spinal fluid; tissue extracts, including cytosolic fraction, S9 fraction and microsomal fraction; aqueous humour; vitreous humour and tissue homogenates.

4) The method of claim 2, wherein the biological matrix is derived from a species selected from the group consisting of one or more of: mouse, rat, monkey, pig, human, dog, guinea pig and rabbit.

5) The method of claim 1, wherein the test fluid comprises at least one purified enzyme.

6) The method of claim 5, wherein the enzyme is selected from the group consisting of snake venom phosphodiesterase and DNAse 1.

7) The method of claim 1, wherein the analyzing step comprises analyzing the mixture using liquid chromatography and mass spectrometry.

8) The method of claim 1, wherein the analyzing step comprises analyzing the mixture using electron spray ionization liquid chromatography mass spectrometry, polyacrylamide gel electrophoresis or capillary electrophoresis to determine a position of the at least one aptamer cleavage site.

9) The method of claim 1, wherein the analyzing step comprises analyzing the mixture using a bioanalytical method selected from the group consisting of one or more of: denaturing polyacrylamide gel electrophoresis, capillary electrophoresis, HPLC, LC/MS, LC/MS/MS, LC/MS/MS/MS, ESI-LC/MS, ESI-LC/MS/MS and ESI-LC/MS/MS/MS.

10) The method of claim 1, wherein the chemical substitution is selected from the group consisting of: a chemical substitution at a sugar position; a chemical substitution at a base position and a chemical substitution at an internucleotide linkage.

11) The method of claim 10, wherein the chemical substitution is selected from the group consisting of: a purine substitution for a pyrimidine; a 2′-deoxy dihydrouridine substitution for a uridine; a 2′-deoxy-5-methyl cytidine for a cytidine; a 2-amino purine substitution for a purine; a phosphorothioate substituted for a phosphodiester; a phosphorodithioate substituted for a phosphodiester; a deoxynucleotide substituted for a 2′-OH nucleotide; a 2′-OMe nucleotide, a 2′-fluoro nucleotide or a 2′-O-methoxyethyl nucleotide substituted for a 2′-OH or deoxynucleotide; the addition of a 3′ cap or a 5′ cap; and the addition of a PEG or PAG polymer.

12) The method of claim 11, wherein the chemical substitution is a phosphorothioate or phosphorodithioate substituted for a phosphodiester.

13) The method of claim 12, wherein the chemical substitution is a phosphorothioate substituted for a phosphodiester.

14) The method of claim 11, wherein the chemical substitution is a 2′-OMe nucleotide, a 2′-fluoro nucleotide or a 2′-methoxyethyl nucleotide substituted for a 2′-OH or deoxynucleotide.

15) The method of claim 14, wherein the chemical substitution is a 2′-OMe nucleotide substituted for a 2′-OH or deoxynucleotide.

16) The method of claim 1, wherein the modified aptamer binds a target with the same or better affinity than the parent aptamer.

17) The method of claim 1, wherein the proximal position comprises a position selected from the group consisting of: a position immediately 5′ to the aptamer cleavage site, a 5′ position at or within three nucleotides of the aptamer cleavage site, a position immediately 3′ to the aptamer cleavage site, a 3′ position at or within three nucleotides of the aptamer cleavage site, and a position at the cleaved internucleotide linkage.

18) The method of claim 17, wherein the proximal position is at the cleaved internucleotide linkage and the chemical substitution is at a phosphate position.

19) The method of claim 17, wherein the proximal position is 5′ or 3′ to the aptamer cleavage site and the chemical substitution is at a sugar or base position.

20) The method of claim 1, wherein the introducing step further comprises introducing more than one chemical substitution at one or more cleavage sites or into a cleavage site or both.

21) The method of claim 1, wherein the method further comprises the step of testing the stability of the modified aptamer in the test fluid.

22) The method of claim 21, comprising repeating the incubating, analyzing, introducing and testing steps iteratively.

23) The method of claim 21, wherein the modified aptamer is more stable in the test fluid than the parent aptamer.

24) The method of claim 21, wherein the stability is assessed by the percent of modified aptamer that remains intact in the test fluid as compared to the percent of the parent aptamer that remains intact in the test fluid.

25) The method of claim 24, wherein the percent of intact aptamer is assessed by a bioanalytical method selected from the group consisting of one or more of: denaturing polyacrylamide gel electrophoresis, capillary electrophoresis, HPLC, LC/MS, LC/MS/MS, LC/MS/MS/MS, ESI-LC/MS, ESI-LC/MS/MS and ESI-LC/MS/MS/MS.

26) The method of claim 23, wherein the modified aptamer is at least 2 fold more stable in the test fluid than the parent aptamer.

27) The method of claim 23, wherein the modified aptamer is at least 5 fold more stable in the test fluid than the parent aptamer.

28) The method of claim 23, wherein the modified aptamer is at least 10′ fold more stable in the test fluid than the parent aptamer.

29) The method of claim 1, wherein the method further comprises the step of determining a dissociation constant of the modified aptamer for a target.

30) The method of claim 29, wherein the method comprises selecting a modified aptamer having a dissociation constant for its target that is the same or less than a dissociation constant of the parent aptamer.

31) The method of claim 1, wherein the modified aptamer binds a target having a biological activity, and wherein the method further comprises the step of measuring the biological activity of the target in the presence and absence of the aptamer.

32) The method of claim 31, wherein the biological activity is measured by an ELISA assay or a cell-based assay.

33) The method of claim 1, wherein the biological activity of the target upon binding the modified aptamer is the same or better than that of the biological activity of the target upon binding the parent aptamer.

34) A method for modifying an aptamer comprising the steps of:
a) incubating a parent aptamer with a biological matrix that contains an enzyme to result in a mixture;
b) analyzing the mixture to identify metabolites of the parent aptamer, thereby detecting at least one aptamer cleavage site in the parent aptamer; and
c) introducing a chemical substitution at a position proximal to the at least one aptamer cleavage site to result in a modified aptamer.

35) A method for enhancing the stability of an aptamer comprising the steps of:
a) incubating a parent aptamer with a test fluid to result in a mixture;
b) analyzing the mixture to identify metabolites of the parent aptamer, thereby detecting at least one aptamer cleavage site in the parent aptamer; and
c) introducing a chemical substitution at a position proximal to the at least one aptamer cleavage site to result in a modified aptamer.

36) A method for enhancing the stability of an aptamer to exonucleases and endonucleases comprising the steps of:
a) incubating a parent aptamer with a test fluid to result in a mixture;
b) analyzing the mixture to identify metabolites of the parent aptamer, thereby detecting at least one aptamer cleavage site in the parent aptamer; and
c) introducing a chemical substitution at a position proximal to the at least one aptamer cleavage site to result in a modified aptamer.
